(19) United States
(12) Patent Application Publication
(10) Pub. No.: US 2017/0007559 A1
(43) Pub. Date: Jan. 12, 2017

(54) COMPOSITIONS AND METHODS FOR PROTECTING COLONIC EPITHELIAL BARRIER FUNCTION

(71) Applicant: The University of Tennessee Research Foundation, Memphis, TN (US)

(72) Inventors: Gabor Jozsef Tigyi, Memphis, TN (US); Radhakrishna Rao, Germantown, TN (US)

(21) Appl. No.: 15/207,907

(22) Filed: Jul. 12, 2016

Related U.S. Application Data
(60) Provisional application No. 62/191,456, filed on Jul. 12, 2015.

(51) Int. Cl. A61K 31/164; A61K 45/06

(52) U.S. Cl. CPC A61K 31/164; A61K 45/06

(57) ABSTRACT

Disclosed are compositions and methods for treating or preventing colonic barrier dysfunction in a human by administering to the human in need thereof a pharmaceutically effective amount of an LPA2 receptor agonist or N-Acetyl L-Cysteine.

### CROSS-REFERENCE TO RELATED APPLICATIONS

This U.S. non-provisional application claims priority to U.S. Provisional Patent Application Nos. 62/191,456 filed on Jul. 12, 2015, the disclosure of which application is incorporated herein by reference.

### GOVERNMENTAL SUPPORT

This invention was made in part with Government support under NIH grants DK55532 and AA12307 to RR. The Government has certain rights in the invention.

### FIELD OF INVENTION

The invention relates to compositions and methods for maintaining colonic barrier function. More specifically, the invention relates to using LPA2 receptor agonists in preventing or treating diseases associated with colonic barrier dysfunctions

### BACKGROUND

The intestine epithelial monolayer constitutes a physical and functional barrier between the organism and the external environment. It regulates nutrients absorption, water and ion fluxes, and represents the first defensive barrier against toxins and enteric pathogens. Epithelial cells are linked together at the apical junctional complex by tight junctions that reduce the extracellular space and the passage of charge entities while forming a physical barrier to lipophilic molecules.

Tight junctions (TJs), the highly specialized intercellular junctions, confer epithelial barrier function in the GI tract (Turner, J. R. Intestinal mucosal barrier function in health and disease. Nat Rev Immunol 9:799-809; 2009). TJs are multi-protein complexes made up of transmembrane proteins such as a occludin, claudins and junctional adhesion molecules, which interact with the intracellular adapter proteins such as ZO-1, ZO-2 and ZO-3 (Anderson, J. M.; Van Itallie, C. M. Physiology and function of the tight junction. Cold Spring Harb Perspect Biol 1:a002584; 2009.). These protein complexes interact with other TJ-specific proteins such as cingulin, AF6, 7H6 and catenins. These protein-protein interactions are essential for the assembly of TJs and maintenance of their integrity. Adherens junctions (AJs), the junctional complexes lie beneath the TJs are also multi protein complexes and composed of transmembrane and adapter proteins, such as E-cadherin and catenins (Baum, B.; Georgiou, M. Dynamics of adherens junctions in epithelial establishment, maintenance, and remodeling. J Cell Biol 192:907-917; 2011.). AJs are not diffusion barriers for macromolecules, but they indirectly regulate the integrity of TJs and therefore the barrier function. TJ and AJ protein complexes interact with the actin cytoskeleton, which support the assembly and maintenance of TJs and AJs (Citalan-Madrid, A. F.; Garcia-Ponce, A.; Vargas-Robles, H.; Betanzos, A.; Schnoor, M. Small GTPases of the Ras superfamily regulate intestinal epithelial homeostasis and barrier function via common and unique mechanisms. Tissue Barriers 1:e26938; 2013.).

Recent observations in humans and in a variety of animal models indicate that an increased intestinal permeability (IP), often referred to as a “leaky gut”, is playing a pathogenic role not only in development of gastrointestinal disorders like inflammatory bowel disease (IBD) and celiac disease, but also in systemic autoimmune diseases, like type 1 diabetes (T1D) (Arrieta M C, Bistritz L, Meddings J B (2006) Alterations in intestinal permeability. Gut 55: 1512-1520. doi: 10.1136/gut.2005.085373; de Kort S, Keszthelyi D, Masclee A A (2011) Leaky gut and diabetes mellitus: what is the link? Obes Rev 12: 449-458. doi: 10.1111/j.1467-789x.2010.00845.x; Fasano A, Shea-Donohue T (2005) Mechanisms of Disease: the role of intestinal barrier function in the pathogenesis of gastrointestinal autoimmune diseases. Nat Clin Pract Gastroenterol Hepatol 2: 416-422. doi: 10.1038/ncpgasthep0259; Visser J, Rozing J, Sapone A, Lammers K, Fasano A (2009) Tight junctions, intestinal permeability, and autoimmunity: celiac disease and type 1 diabetes paradigms. Ann N Y Acad Sci 1165: 195-205. doi: 10.1111/j.1749-6632.2009.04037.x).

Disruption of intestinal epithelial barrier function plays an important role in causing endotoxemia in alcoholics. Intestinal flora is confined to the colon and the distal ileum. However, colonic epithelial barrier dysfunction is poorly understood in the art. One objective of this present application is to understand the causation of colonic barrier dysfunction and discover compositions and methods to prevent or treat colonic epithelial barrier dysfunction.

### BRIEF SUMMARY OF THE INVENTION

In one aspect, the disclosed invention is a method of treating or preventing colonic barrier dysfunction in a human by administering to the human in need thereof a pharmaceutically effective amount of an LPA2 receptor agonist. The LPA2 receptor agonist can be RP-1, or LPA.

In some embodiments, the colonic barrier dysfunction is caused by irradiation. In other embodiments, the colonic barrier dysfunction is caused by alcohol consumption.

In some embodiments, the administration of the compound is prior to the occurrence of the colonic barrier dysfunction. In other embodiments, the administration of the compound is after the occurrence of the colonic barrier dysfunction.

In another aspect, the disclosed invention is directed to a medicament for treating or preventing colonic barrier dysfunction in a human, comprising an LPA2 receptor agonist. In some embodiments, the colonic barrier dysfunction is caused by irradiation. In other embodiments, the colonic barrier dysfunction is caused by alcohol consumption.

In yet another aspect, the disclosed invention is a method of treating or preventing colonic barrier dysfunction in a human, comprising administering to the human in need thereof a pharmaceutically effective amount of N-Acetyl L-Cysteine. In some embodiments, the colonic barrier dysfunction is caused by irradiation. In other embodiments, the colonic barrier dysfunction is caused by alcohol consumption.

In some embodiments, the administration of the compound is prior to the occurrence of the colonic barrier dysfunction. In other embodiments, the administration of the compound is after the occurrence of the colonic barrier dysfunction.

In a further aspect, the disclosed invention is a medicament for treating or preventing colonic barrier dysfunction in a human, comprising N-Acetyl L-Cysteine. In some embodiments, the colonic barrier dysfunction is caused by irradiation. In other embodiments, the colonic barrier dysfunction is caused by alcohol consumption.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A and FIG. 1B show photos supporting the notion that ionizing radiation induces rapid redistribution of TJ proteins in mouse intestine. Mice were subjected to total body irradiation (IR) or sham treatment (Sham). At 2-24 hours post irradiation (Post-IR), cryosections of distal colon (1A) and ileum (1B) were stained for occludin (green in FIG. 1A and red in FIG. 1B) and ZO-1 (red in 1A and green in 1B) by immunofluorescence method, and the nucleus stained with Hoechst 33342 (blue). Fluorescence images were collected by confocal microscopy.

FIG. 2A and FIG. 2B show photos supporting the notion that ionizing radiation induces rapid redistribution of Cldn-3 and reorganization of actin cytoskeleton. Mice were subjected to total body irradiation (IR) or sham treatment (Sham). At 2-24 hours post irradiation (Post-IR), cryosections of distal colon (2A) and ileum (2B) were stained for Cldn-3 (red) and F-actin (green) by immunofluorescence method, and the nucleus stained with Hoechst 33342 (blue). Fluorescence images were collected by confocal microscopy.

FIG. 3A and FIG. 3B show photos supporting the notion that ionizing radiation induces rapid redistribution of AJ proteins in mouse intestine. Mice were subjected to total body irradiation (IR) or sham treatment (Sham). At 2-24 hours post irradiation (Post-IR), cryosections of distal colon (3A) and ileum (3B) were stained for E-cadherin (green) and β-catenin (red) by immunofluorescence method, and the nucleus stained with Hoechst 33342 (blue). Fluorescence images were collected by confocal microscopy.

FIG. 4A and FIG. 4B show photos supporting the notion that NAC feeding blocks radiation-induced redistribution of TJ proteins in mouse intestine. Mice were fed a liquid diet with or without 20 mM NAC for 5 days prior to total body irradiation (IR) or sham treatment (Sham). At 2 hours post irradiation (Post-IR), cryosections of distal colon (4A) and ileum (4B) were stained for occludin (green) and ZO-1 (red) by immunofluorescence method, and the nucleus stained with Hoechst 33342 (blue). Fluorescence images were collected by confocal microscopy.

FIG. 5A and FIG. 5B show photos supporting the notion that NAC feeding blocks radiation-induced redistribution of Cldn-3 and reorganization of actin cytoskeleton. Mice were fed a liquid diet with or without 20 mM NAC for 5 days prior to total body irradiation (IR) or sham treatment (Sham). At 2 hours post irradiation (Post-IR), cryosections of distal colon (5A) and ileum (5B) were stained for F-actin (green) and Cldn-3 (red) by immunofluorescence method, and the nucleus stained with Hoechst 33342 (blue). Fluorescence images were collected by confocal microscopy.

FIG. 6A and FIG. 6B show photos supporting the notion that NAC feeding blocks radiation-induced redistribution of AJ proteins in mouse intestine. Mice were fed a liquid diet with or without 20 mM NAC for 5 days prior to total body irradiation (IR) or sham treatment (Sham). At 2 hours post irradiation (Post-IR), cryosections of distal colon (6A) and ileum (6B) were stained for E-cadherin (green) and β-catenin (red) by immunofluorescence method, and the nucleus stained with Hoechst 33342 (blue). Fluorescence images were collected by confocal microscopy.

FIGS. 7A, 7B, 7C, 7D, 7E, and 7F show photos and graphs supporting the notion that NAC feeding blocks radiation-induced depletion of AJ proteins in mouse intestine. Mice were fed a liquid diet with or without 20 mM NAC for 5 days prior to total body irradiation (IR) or sham treatment (Sham). At 2 hours post irradiation (Post-IR), Triton-insoluble fractions were prepared from the distal colonic mucosa and immunoblotted for TJ and AJ proteins (7A). Immunoblot bands for occludin (7B), ZO-1 (FIG. 7C), Cldn-3 (7D), E-cadherin (7E) and β-catenin (7F) were quantitated by densitometric analysis and normalized to band density of corresponding actin bands. Values are mean+SE (n=3). Asterisks indicate the values that are significantly (p<0.05) different from values for corresponding control group, and the hash tags indicate the values that are significantly (p<0.05) different from values for corresponding IR group.

FIGS. 8A and 8B show graphs supporting the notion that NAC feeding blocks radiation-induced mucosal barrier dysfunction in mouse intestine. Adult mice were fed liquid diet with or without 20 mM N-acetyl L-cysteine (NAC) for 5 days prior to total body irradiation (IR) or sham treatment (Sham). At two hours post irradiation, intestinal mucosal barrier function evaluated by measuring vascular-to-luminal flux of FITC-inulin in vivo (8A) as well as in vitro (8B) as described in Methods. 8A: In vivo flux values are mean±sem (n=6). Asterisk indicates the value that is significantly (p<0.05) different from corresponding value for Sham-treated mice, and the “#” indicates the value that is significantly different from corresponding value for IR group. 8B: At 3 hours post irradiation inulin absorption from the lumen of colonic loops was measured. Values for absorbed fluorescence are mean±sem (n=5). Asterisk indicates the value that is significantly (p<0.05) different from corresponding value for Sham-treated mice, and the “#” indicates the value that is significantly different from corresponding value for IR group.

FIGS. 9A, 9B, 9C and 9D show photos and graphs supporting the notion that NAC feeding blocks radiation-induced protein thiol oxidation in mouse intestine. Adult mice were fed liquid diet with or without 20 mM N-acetyl L-cysteine (NAC) for 5 days prior to total body irradiation (IR) or sham treatment (Sham). At two hours post irradiation, the levels of reduced and oxidized protein thiols in distal colon (9A & 9B) and ileum (9C & 9D) were measured as described in Methods. Fluorescence images were collected by confocal microscopy (9A & 9C). Fluorescence density was evaluated by using Image J software (9B & 9D). Values are mean±sem (n=5). Asterisk indicates the value that is significantly (p<0.05) different from corresponding value for Sham-treated mice, and the “#” indicates the value that is significantly different from corresponding value for IR group.

FIGS. 10A, 10B, 10C, 10D and 10E show photos and graphs supporting the notion that radiation induces redistribution of TJ proteins and barrier dysfunction in Caco-2 and m-ICC12 cell monolayers. Caco-2 (10A-10D) or m-ICC12 (10E) cell monolayers were irradiated (IR, 2 or 4 Gy) with or without NAC (10 mM) pretreatment for one hour. Inulin permeability (10A) and transepithelial electrical resistance (TER) (10B) were measured. Fixed cell monolayers were stained for occludin and/or ZO-1. Merged images in 10C show occludin (green) and ZO-1 (red). Fluorescence for ZO-1 (10D) was measured by densitometry. Values are mean±sem (n=6). Each “n” in 10D is average of fluorescence from 6 spots in the same monolayers. Asterisk indicates values that are significantly (p<0.05) different from corresponding sham values, and symbol # indicates values different from corresponding IR values.

FIGS. 11A and 11B show photos supporting the notion that radiation induces cofilin activation. 11A: Mice were subjected to PBI-BM5 (16 Gy) and after 24 hours injected with RP-1 or vehicle (Veh). Colon sections were stained for F-actin and Cofilin^{pS3} (cofilin phosphorylated on Serine on position 3 is inactive) 52 hours post irradiation. Merged images show actin (green), cofilin^{pS3} (red) and nucleus (blue). 11B: Caco-2 cell monolayers were irradiated (4 Gy) with or without LPA pretreatment, one hour later stained for F-actin (green) and cofilin^{pS3} (red).

FIGS. 12A, 12B, 12C and 12D show photos and graphs supporting the notion that RP-1 blocks TBI-induced redistribution of TJ and AJ proteins. Two hours after TBI (4 Gy) with or without RP-1 pretreatment colonic sections were stained for occludin (green) and ZO-1 (red) (12A & 12B) or E-cadherin (green) and β-catenin (red) (12C & 12D) proteins; blue is nucleus in both panels. Fluorescence for ZO-1 (12B) and β-catenin (12D) was measured by densitometry. Values are mean±sem (n=3); each “n” is average of fluorescence from 6 spots in the same monolayers. Asterisk indicates values that are significantly (p<0.05) different from corresponding sham values, and symbol # indicates values different from corresponding control values.

FIGS. 13A, 13B, 13C, 13D, 13E and 13F show photos and graphs supporting the notion that RP-1 mitigates PBI-BM5-induced redistribution of TJ and AJ proteins. Mice were subjected to PBI-BM5 (16 Gy) and after 24 hours injected with RP-1 or vehicle. Colon sections were stained for different proteins 52 hours post irradiation. 13A: Occludin (green), ZO-1 (red) and nucleus (blue) at 52 hr post-IR. 13B: Densitometry of ZO-1 fluorescence. 13C: E-cadherin (green), β-catenin (red) and nucleus (blue) at 52 hr post-IR. 13D: Densitometry of E-cadherin fluorescence; 52 hr. 13E: Reduced and oxidized protein thiol stain. 13F: F-actin (green), Nrf2 (red) and nucleus (blue). Values in 13B and 13D are mean±sem (n=4); each “n” is average of fluorescence from 6 spots in the same monolayers. Asterisk indicates values that are significantly (p<0.05) different from corresponding sham values, and symbol # indicates values different from corresponding IR values. In 13E & 13F, similar results in two other mice per group.

FIGS. 14A, 14B and 14C show photos and graphs supporting the notion that LPA attenuates radiation-induced redistribution of TJ proteins in Caco-2 and m-ICC12 cell monolayers. Cell monolayers were treated with or without LPA prior to irradiation. One hour later cell monolayers were stained for TJ proteins. 14A: Caco-2 cells, stained for occludin (green) and ZO-1 (red). 14B: Densitometry of ZO-1 fluorescence in Caco-2 cells. 14C: m-ICC12 cells stained for ZO-1.

FIG. 15 shows photos and graphs supporting the notion that RP-1 administration blocks chronic+binge ethanol-induced increase in inulin permeability in mouse intestine. Adult female mice were fed 5% ethanol in Lieber-DeCarli liquid diet for 10 days followed by one-time gavage of ethanol (5 g/kg BW) to model a chronic+binge model of alcohol consumption. Intestinal permeability was evaluated by measuring vascular-to-luminal flux of FITC-inulin. Values are mean±SE. Asterisk indicates the value that is significantly different (P<0.05) from the value for Pair fed group, and the symbol # indicates the value different from the Ethanol fed+Carrier group.

FIG. 16 shows photos and graphs supporting the notion that RP1 administration ameliorates fat accumulation in liver by chronic+binge ethanol feeding in mice. Adult female mice were fed 5% ethanol in Lieber-DeCarli liquid diet for 10 days followed by one time gavage of ethanol (5 g/kg BW) to model a chronic+binge model of alcohol consumption. Liver triglyceride was measured. Values are mean±SE. Asterisk indicates the value that is significantly different (P<0.05) from the value for Pair fed group, and the symbol # indicates the value different from the Ethanol fed+Carrier group.

FIG. 17 shows photos and graphs supporting the notion that LPA2 deficiency enhances chronic+binge ethanol-induced inulin permeability in mouse colon. Adult wild type (WT) and LPA2 knockout (KO) mice were fed 5% ethanol in Lieber-DeCarli liquid diet for 10 days followed by one-time gavage of ethanol (5 g/kg BW) to model a chronic+binge model of alcohol consumption. Intestinal permeability was evaluated by measuring vascular-to-luminal flux of FITC-inulin. Values are mean±SE. Asterisk indicates the value that is significantly different (P<0.05) from corresponding values for WT group.

FIG. 18 shows photos and graphs supporting the notion that LPA2 deficiency enhances fat accumulation in liver by chronic+binge ethanol feeding in mice. Adult wild type (WT) and LPA2 knockout (KO) mice were fed ethanol in Lieber-DeCarli liquid diet for 10 days followed by one time gavage of ethanol (5 g/kg BW) to model a chronic+binge model of alcohol consumption. Control animals were pair fed (PF) isocaloric diet without ethanol. Liver triglyceride was measured. Values are mean±SE. Asterisk indicates the value that is significantly different (P<0.05) from corresponding values for WT group.

FIG. 19 shows photos and graphs supporting the notion that LPA2 deficiency enhances fat accumulation in liver by chronic ethanol feeding in mice. Adult wild type (WT) and LPA2 knockout (KO) mice were fed 1-6% ethanol (EF) in Lieber-DeCarli liquid diet for 4 weeks days to model chronic alcohol consumption. Control animals were pair fed (PF) isocaloric diet without ethanol. Liver triglyceride was measured. Values are mean±SE. Asterisk indicates the value that is significantly different (P<0.05) from corresponding values for WT group.

FIGS. 20A and 20B show photos and graphs supporting the notion that LPA treatment attenuates ethanol+acetaldehyde (E+A)-induced decrease in transepithelial electrical resistance (TER) and increase in inulin permeability in Caco-2 cell monolayers. Caco-2 cell monolayers were pretreated with or without LPA (10 μM) for 20 min prior to exposure to ethanol (0.5%) and acetaldehyde (400 μM) for 4 hours. Unidirectional flux of FITC-inulin (20A) and transepithelial electrical resistance (TER) (20B) were measured. Values mean±SE (n=6). Asterisks indicate the values that are significantly (P<0.05) different from corresponding control values. The symbol # indicates the values that are different (P<0.05) from corresponding E+A value for carrier group.

### DETAILED DESCRIPTION OF THE INVENTION

The invention is based on the surprising findings that colonic epithelial barrier dysfunction may be restored or prevented by various compounds. As such the present application provides compositions and methods for preventing and treating colonic barrier dysfunctions.

In some embodiments, colonic epithelial barrier dysfunction may be caused by radiotherapy or accidental exposure to ionizing radiation. Radiotherapy or accidental exposure to ionizing radiation causes severe damage to healthy tissues. The gastrointestinal (GI) tract is one of the radiation-sensitive organs in the body, and the GI complications of radiation are collectively referred to as GI acute radiation syndrome or GI-ARS (Macia, I. G. M.; Lucas Calduch, A.; Lopez, E. C. Radiobiology of the acute radiation syndrome. Rep Pract Oncol Radiother 16:123-130; 2011.). GI-ARS is characterized by nausea and diarrhea during the early stage of radiation injury, and endotoxemia and bacteremia leading to septicemia in the later stage (Dubois, A.; Walker, R. I. Prospects for management of gastrointestinal injury associated with the acute radiation syndrome. Gastroenterology 95:500-507; 1988; Harb, A. H.; Abou Fadel, C.; Sharara, A. I. Radiation enteritis. Curr Gastroenterol Rep 16:383; 2014; Wang, A.; Ling, Z.; Yang, Z.; Kiela, P. R.; Wang, T.; Wang, C.; Cao, L.; Geng, F.; Shen, M.; Ran, X.; Su, Y.; Cheng, T.; Wang, J. Gut microbial dysbiosis may predict diarrhea and fatigue in patients undergoing pelvic cancer radiotherapy: a pilot study. PLoS One 10:e0126312; 2015; Damman, C. J.; Surawicz, C. M. The gut microbiota: a microbial arsenal protecting us from infectious and radiation-induced diarrhea. Gastroenterology 136:722-724; 2009.) Total body irradiation (TBI) causes ablation of crypt cell proliferation and apoptosis leading to GI mucositis (Somosy, Z.; Horvath, G.; Telbisz, A.; Rez, G.; Palfia, Z. Morphological aspects of ionizing radiation response of small intestine. Micron 33:167-178; 2002.). The prevailing concept in this field is that the threshold for GI-ARS is 5-10 Gy (Driak, D.; Osterreicher, J.; Vavrova, J.; Rehakova, Z.; Vilasova, Z. Morphological changes of rat jejunum after whole body gamma-irradiation and their impact in biodosimetry. Physiol Res 57:475-479; 2008.); it is greater than 10 Gy according to the Fact Sheet for Physicians by Center for Disease Control. Whereas the small intestine is the primary target of radiation, the colon is relatively resistant to radiation injury (Freeman, S. L.; Hossain, M.; MacNaughton, W. K. Radiation-induced acute intestinal inflammation differs following total-body versus abdominopelvic irradiation in the ferret. Int J Radiat Biol 77:389-395; 2001; Cameron, S.; Schwartz, A.; Sultan, S.; Schaefer, I. M.; Hermann, R.; Rave-Frank, M.; Hess, C. F.; Christiansen, H.; Ramadori, G. Radiation-induced damage in different segments of the rat intestine after external beam irradiation of the liver. Exp Mol Pathol 92:243-258; 2012; Potten, C. S.; Grant, H. K. The relationship between ionizing radiation-induced apoptosis and stem cells in the small and large intestine. Br J Cancer 78:993-1003; 1998.) The facts that endotoxemia and bacteremia are important events in the pathogenesis of ARS and the colon is the primary source of endotoxins highlight the critical, yet so far unrealized importance of colonic tissue injury in the pathogenesis of GI-ARS. Furthermore, colitis is one of clinical problems associated with radiotherapy that predominantly affects gut microflora and induces colonic endotoxemia (Harb, A. H.; Abou Fadel, C.; Sharara, A. I. Radiation enteritis. Curr Gastroenterol Rep 16:383; 2014.). Dysbiosis, infection and endotoxemia involve disruption of the structural and functional integrity of gut mucosa, which affects selective permeability of important nutrients as well as endotoxins (Naftalin, R. Alterations in colonic barrier function caused by a low sodium diet or ionizing radiation. J Environ Pathol Toxicol Oncol 23:79-97; 2004; Nejdfors, P.; Ekelund, M.; Westrom, B. R.; Willen, R.; Jeppsson, B. Intestinal permeability in humans is increased after radiation therapy. Dis Colon Rectum 43:1582-1587; discussion 1587-1588; 2000.).

In other embodiments, colonic epithelial barrier dysfunction may be caused by alcohol consumption. It is known that alcohol consumption leads to increased intestinal permeability and endotoxemia, which are involved in the pathogenesis of alcoholic liver disease (ALD). Intestinal microflora and the generation of acetaldehyde in the colonic lumen play crucial roles in alcoholic intestinal permeability and endotoxemia. Evidence indicates that intestinal microflora not only is the source of circulating endotoxins but also plays a role in the generation and accumulation of acetaldehyde in the colonic lumen and has a subsequent influence on epithelial barrier dysfunction. See, R K Rao, Hepatology, 2009, 638-644.

In one aspect, the invention is directed to methods of treating colonic epithelial barrier dysfunction with a pharmaceutically effective amount of an LPA2 receptor agonist.

In some embodiments, LPA2 receptor-specific agonists are lipid-like ligands, primarily to address the hydrophobic environment of the S1P and LPA G protein-coupled receptor (GPCR) ligand binding pockets. In some embodiments, LPAs receptor ligands are nonlipid ligands, e.g., Ki16425 (Ohta et al., Ki16425, a subtype-selective antagonist for EDG-family lysophosphatidic acid receptors. Mol Pharmacol (2003) 64(4): 994-1005). In other embodiments, LPA2 receptor-specific agonists are non-lipid like benzoic acid derivatives that are described in published patent application US 2014/0057936, which is incorporated herein by reference in its entirety. In some embodiments, the LPA2 receptor-specific agonist is RP-1. In other embodiments, LPA2 receptor-specific agonist is LPA.

In another aspect, the invention is directed to methods of treating colonic epithelial barrier dysfunction with a pharmaceutically effective amount of N-Acetyl L-Cysteine.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals.

In some embodiments, two or more LPA2 receptor agonists are administered simultaneously or sequentially. In other embodiments, NAC and one or more LPA2 receptor agonists are administered simultaneously or sequentially.

Alternatively, the compounds can be administered as a sustained release formulation, in which case less frequent administration is required. Dosage and frequency vary depending on the half-life of the compound in the patient.

The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated or until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective amount” of the compound can result in a decrease in severity of disease symptoms, an increase in frequency and duration of disease symptom-free periods, or a prevention of impairment or disability due to the compromised colonic epithelial barrier dysfunction.

A composition of the present invention can be administered by one or more routes of administration using one or more of a variety of methods known in the art. As will be appreciated by the skilled artisan, the route and/or mode of administration will vary depending upon the desired results. Routes of administration for antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, a compound can be administered by a nonparenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Pharmaceutical Compositions

In another aspect, the present invention provides a pharmaceutical composition for treating or preventing colonic epithelial barrier dysfunction in a patient, which composition may comprise a LPA2 receptor agonist or NAC. In some embodiment, the composition comprises two or more LPA2 receptor agonists. In other embodiments, the composition comprises NAC. In still other embodiments, the composition comprises NAC and one or more LPA2 receptor agonists. The pharmaceutical composition may be formulated with a pharmaceutically acceptable carrier.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). Depending on the route of administration, the active compound, i.e., antibody, immunoconjugate, or bispecific molecule, may be coated in a material to protect the compound from the action of acids and other natural conditions that may inactivate the compound.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, from about 0.1 percent to about 70 percent, or from about 1 percent to about 30 percent of active ingredient in combination with a pharmaceutically acceptable carrier.

Therapeutic compositions can be administered with medical devices known in the art. For example, in one embodiment, a therapeutic composition of the invention can be administered with a needleless hypodermic injection device, such as the devices shown in U.S. Pat. Nos. 5,399,163; 5,383,851; 5,312,335; 5,064,413; 4,941,880; 4,790,824 or 4,596,556. Examples of well-known implants and modules useful in the present invention include: U.S. Pat. No. 4,487,603, which shows an implantable micro-infusion pump for dispensing medication at a controlled rate; U.S. Pat. No. 4,486,194, which shows a therapeutic device for administering medicants through the skin; U.S. Pat. No. 4,447,233, which shows a medication infusion pump for delivering medication at a precise infusion rate; U.S. Pat. No. 4,447,224, which shows a variable flow implantable infusion apparatus for continuous drug delivery; U.S. Pat. No. No. 4,439,196, which shows an osmotic drug delivery system having multi-chamber compartments; and U.S. Pat. No. 4,475,196, which shows an osmotic drug delivery system. These patents are incorporated herein by reference. Many other such implants, delivery systems, and modules are known to those skilled in the art.

In certain embodiments, the human monoclonal antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade, 1989 J. Cline Pharmacol. 29:685). Exemplary targeting moieties include folate or biotin (see, e.g., U.S. Pat. No. 5,416,016 to Low et al.); mannosides (Umezawa et al., 1988 Biochem. Biophys. Res. Commun. 153:1038); antibodies (P. G. Bloeman et al., 1995 FEBS Lett. 357:140; M. Owais et al., 1995 Antimicrob. Agents Chemother. 39:180); surfactant protein A receptor (Briscoe et al., 1995 Am. J. Physiol 1233:134); p120 (Schreier et al., 1994 J. Biol. Chem. 269:9090); see also K. Keinanen; M. L. Laukkanen, 1994 FEBS Lett. 346:123; J. J. Killion; I. J. Fidler, 1994 Immunomethods 4:273.

### EXAMPLE 1

### Ionizing Radiation Rapidly Disrupts Intestinal Epithelial Tight Junctions and Barrier Function in Mouse Colon In Vivo, and Protection of Colonic Barrier Function by N-Acetyl L-Cysteine

In this example, the inventors demonstrated that radiation rapidly disrupts TJs, AJs and the actin cytoskeleton that can be prevented by N-acetyl L-cysteine (NAC). These radiation-induced changes in the colon are herein designated as the “Colonic Radiation Sub-syndrome (CRS)”. Specifically, the inventors provided data demonstrating that: radiation caused a redistribution of TJ proteins, occludin, ZO-1 and claudin-3 (Cldn3), the AJ proteins, E-cadherin and β-catenin, as well as the actin cytoskeleton as early as 2 hours post-irradiation and this effect sustained for at least 24 hours; feeding NAC prior to irradiation blocked radiation-induced disruption of TJs, AJs and the actin cytoskeleton; radiation increased mucosal permeability to inulin in colon, which was prevented by NAC feeding NAC; the levels of reduced protein thiols in colon were dramatically reduced by radiation with a concomitant increase in the levels of oxidized protein thiol; radiation-induced protein thiol oxidation was blocked by NAC feeding; and analysis of ileum in these experiments also showed a rapid disruption of TJs, AJs and the actin cytoskeleton, which was blocked by NAC pretreatment.

1.1. Methods and Materials

1.1.1 Chemicals

Maltose dextrin was purchased from Bioserv (Flemington, N.J.). Regular Lieber DeCarli ethanol diet (Dyet #710260) was purchased from Dyets Inc. (Bethlehem, Pa.). Hoechst 33342 dye and BODIPY FL-N-(2-aminoethyl) maleimide were purchased from Life technologies (Grand Island, N.Y.). N-ethylmaleimide and tris (2-carboxyethyl) phosphine were from Sigma Aldrich (St. Louis, Mo.). All other chemicals were purchased from either Sigma Aldrich (St. Louis, Mo.) or Thermo Fisher Scientific (Tustin, Calif.).

1.1.2 Antibodies

Anti-ZO-1, anti-occludin, and anti-Claudin-3 (Cldn-3) antibodies were purchased from Invitrogen (Carlsbad, Calif.). Anti-E-Cadherin and anti-□-catenin antibodies were purchased from BD Biosciences (Billerica, Mass.). Horseradish peroxidase-conjugated anti-mouse IgG and anti-rabbit IgG, and anti-□-actin antibodies were obtained from Sigma Aldrich (St. Louis, Mo.). AlexaFlour-488-conjugated anti-mouse IgG and Cy3-conjugated anti-rabbit IgG were purchased from Molecular Probes (Eugene, Oreg.)

1.1.3 Animals and Diets

Female C57BL/6 mice (12-14 weeks, Harlan Laboratories, Houston, Tex.) were used for all experiments. All animal experiments were performed according to the protocols approved by UTHSC Institutional Animal Care and Use Committee. Animals were housed in institutional animal care facility with 12 hours light and dark cycles. All mice had free access to regular laboratory chow and water until the start of experiments.

1.1.4 Study Protocol

In the first study, adult female (12-14 week's age) mice were subjected to total body irradiation (4 Gy). At 2-24 hours post-irradiation (IR), the integrity of colonic epithelial TJs, AJs and actin cytoskeleton was examined. In a second study, mice were randomized to four groups: Sham (control), IR, NAC and NAC+IR, and fed a liquid diet with (NAC and NAC+IR) or without (Sham and IR) 20 mM NAC for 5 days prior to irradiation. IR and NAC+IR mice were subjected to total body irradiation (4 Gy), while Sham and NAC mice were sham-treated. At 2 hours post-irradiation, colon and ileum were collected and examined for oxidative stress and epithelial junctional integrity. At the end of experiment, gut permeability was measured as described below. Colon and ileum segments were stored frozen for further analyses.

1.1.5 Gut Permeability In Vivo and In Vitro

Mucosal barrier dysfunction was evaluated by measuring gut permeability to FITC-inulin (6 kDa). On the last day of experiment, mice were intravenously injected with FITC-inulin (50 mg/ml solution; 2 μl/g body weight) via tail vein. One hour after injection, blood samples were collected by cardiac puncture under isoflurane anesthesia for plasma preparation. Luminal contents from colon and ileum were flushed with 0.9% saline. Fluorescence in plasma and luminal flushing was measured using fluorescence plate reader. Fluorescence values in the luminal flushing were normalized to fluorescence values in corresponding plasma samples and calculated as percent of amount injected.

At 3 hours post irradiation, colonic loops were prepared and filled with 0.15 ml of 0.9% saline containing FITC-inulin (0.05 mg/ml) and incubated for 75 min in an incubator. Luminal contents were measured for fluorescence to evaluate inulin absorption from colonic lumen.

1.1.6 Immunofluorescence Microscopy

Cryo-sections of colon (10 μm thickness) and ileum (12 μm thickness) were fixed in acetone methanol mixture (1:1) at 20° C. for 2 min and rehydrated in phosphate buffered saline (PBS). Sections were permeabilized with 0.5% Triton X-100 in PBS for 15 min and blocked in 4% non-fat milk in TBST (20 mM Tris, pH 7.2 and 150 mM NaCl). It was then incubated for one hour with primary antibodies (mouse monoclonal anti-occludin and rabbit polyclonal anti-ZO-1 antibodies or mouse monoclonal E-cadherin and rabbit polyclonal anti-β-catenin antibodies), followed by incubation for one hour with secondary antibodies (AlexaFluor-488-conjugated anti-mouse IgG and Cy3-conjugated anti-rabbit IgG antibodies from Molecular Probes, Eugene, Oreg.) containing Hoechst 33342. The fluorescence was examined by using a confocal microscope (Zeiss 710) and images from x-y sections (1 μm) were collected using Zen software. Images were stacked using the Image J software (NIH) and processed by Adobe Photoshop (Adobe Systems Inc., San Jose, Calif.).

1.1.7 Preparation of Detergent-Insoluble Fraction

Actin-rich detergent-insoluble fraction was prepared as described previously (Elias, B. C.; Suzuki, T.; Seth, A.; Giorgianni, F.; Kale, G.; Shen, L.; Turner, J. R.; Naren, A.; Desiderio, D. M.; Rao, R. Phosphorylation of Tyr-398 and Tyr-402 in occludin prevents its interaction with ZO-1 and destabilizes its assembly at the tight junctions. J Biol Chem 284:1559-1569; 2009; Kale, G.; Naren, A. P.; Sheth, P.; Rao, R. K. Tyrosine phosphorylation of occludin attenuates its interactions with ZO-1, ZO-2, and ZO-3. Biochem Biophys Res Commun 302:324-329; 2003.). Mucosal scrapping from colon and ileum were incubated on ice for 15 min with lysis buffer-CS (Tris buffer containing 1% Triton-X100, 2 μg/ml leupeptin, 10 μg/ml aprotinin, 10 μg/ml bestatin, 10 μg/ml pepstatin-A, 10 μl/ml of protease inhibitor cocktail, 1 mM sodium vanadate and 1 mM PMSF). Briefly, mucosal lysates were centrifuged at 15,600×g for 4 min at 4° C. to sediment the high-density actin-rich detergent-insoluble fraction. The pellet was suspended in 100 μl of preheated lysis buffer-D (20 mM Tris buffer, pH 7.2, containing 10 μl/ml of protease inhibitor cocktail, 10 mM sodium fluoride, 1 mM sodium vanadate and 1 mM PMSF) and sonicated to homogenize the actin cytoskeleton, and heated at 100° C. Protein content was measured by BCA method (Pierce Biotechnology, Rockford, Ill.). Triton-insoluble and soluble fractions were mixed with equal volume of Laemmli's sample buffer (2× concentrated), heated at 100° C. for 5 min and 25-40 μg protein samples was used for immunoblot analysis.

1.1.8 Immunoblot Analysis

Triton soluble and insoluble fractions were separated by SDS-polyacrylamide gel (7%) electrophoresis and transferred to PVDF membranes as described before (Samak, G.; Chaudhry, K. K.; Gangwar, R.; Narayanan, D.; Jaggar, J. H.; Rao, R. Calcium/Ask1/MKK7/JNK2/c-Src signaling cascade mediates disruption of intestinal epithelial tight junctions by dextran sulfate sodium. Biochem J 465:503-515; 2015.). Membranes were immunoblotted for different proteins using specific antibodies for different tight junction and adherens junction proteins with β-actin as house keeping protein in combination with HRP-conjugated anti-mouse IgG or anti-rabbit IgG secondary antibodies. The blots were developed using ECL chemiluminescence method (Pierce) and quantitated by densitometry using Image J software. The density for each band was normalized to density of corresponding actin band.

1.1.9 Protein Thiol Assay

Protein thiols in colonic sections were assessed as described before [23]. Reduced protein thiols were evaluated by staining cryosections colon with BODIPY FL-N-(2-aminoethyl) maleimide (Flm) and confocal microscopy at excitation and emission wavelengths, 490 nm and 534 nm, respectively. For oxidized protein thiols, the reduced protein thiol was first alkylated with N-ethylmaleimide followed by reduction of oxidized protein thiols with tris (2-carboxyethyl) phosphine prior to staining with Flm. Control staining is done after N-ethylmaleimide treatment. Fluorescence images collected and fluorescence quantitated by Image J software.

1.1.10 Statistical Analyses

Values are expressed as mean±SE of 4-8 animals. Statistical analysis was performed by Student's t-test. Statistical significance assessed at p-values<0.05.

1.2. Results

1.2.1 Ionizing Radiation Induces a Rapid Disruption of Tight Junctions and Reorganization of Actin Cytoskeleton in the Intestinal Epithelium.

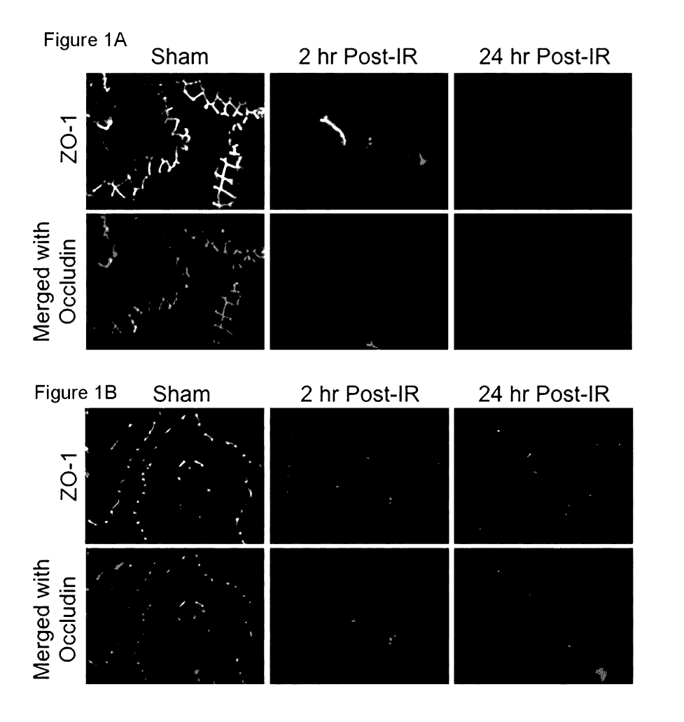

A disruption of epithelial TJs without morphological or cellular damage may lead to increase in paracellular permeability and endotoxin flux into the mucosa. We investigated the effect of mild radiation (4 Gy) on the intestinal epithelial TJs, AJs and the actin cytoskeleton at varying time points. Confocal microscopy of colon (FIG. 1A) and ileum (FIG. 1B) showed a co-localization of occludin and ZO-1 at the intercellular junctions of epithelial cells. Radiation induced a redistribution of both occludin and ZO-1 from the intercellular junctions into the intracellular compartment as early as 2 hours post-irradiation. This effect of radiation sustained for at least 24 hours in both colon (FIG. 1A) and ileum (FIG. 1B). Redistribution of occludin and ZO-1 appeared to be much severe in colon compared to that in ileum even at 2 hours post irradiation.

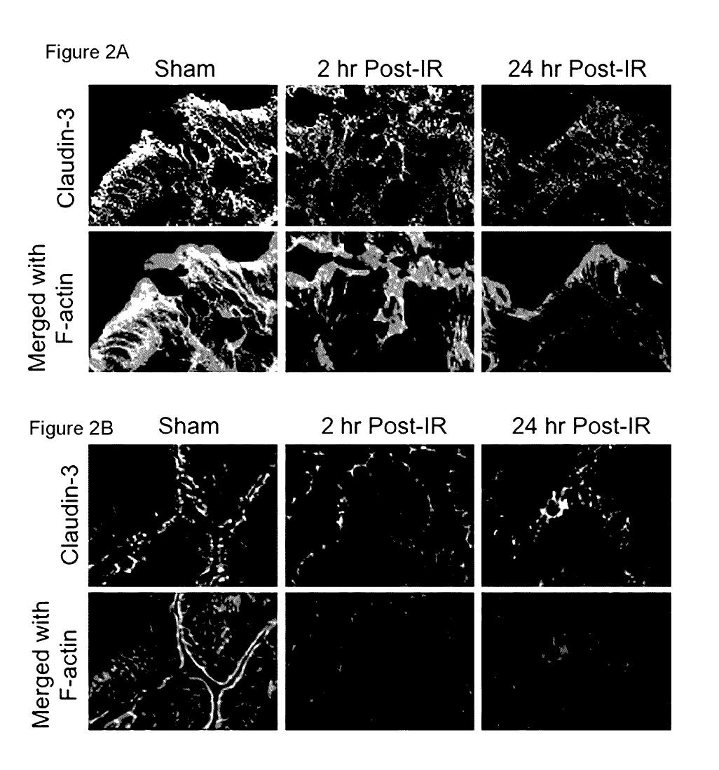

Claudins are a set of transmembrane proteins of TJs that play a crucial role in TJ assembly and maintenance of barrier function [24]. Our data show that Cldn-3 is localized predominantly at the intercellular junctions of the colonic (FIG. 2A) and ileal (FIG. 2B) epithelium. Radiation induced a loss of junctional distribution of Cldn-3 in both colon and ileum, but once again, damage appeared to be more severe in the colon. Staining for F-actin showed that actin cytoskeleton is organized in the colonic (FIG. 2A) and ileal (FIG. 2B) epithelia with distinct distributions at the apical microvilli and the lateral sub-membrane regions. Radiation induced a time-dependent loss of F-actin structure in the colonic epithelium (FIG. 2A), suggesting a disassembly of actin cytoskeleton. Similarly, radiation reduced F-actin levels in the epithelium of ileum (FIG. 2B); the effect on F-actin appeared to be more severe in the ileum compared to that in colon.

1.2.2 Ionizing Radiation Induces a Rapid Disruption of AJs.

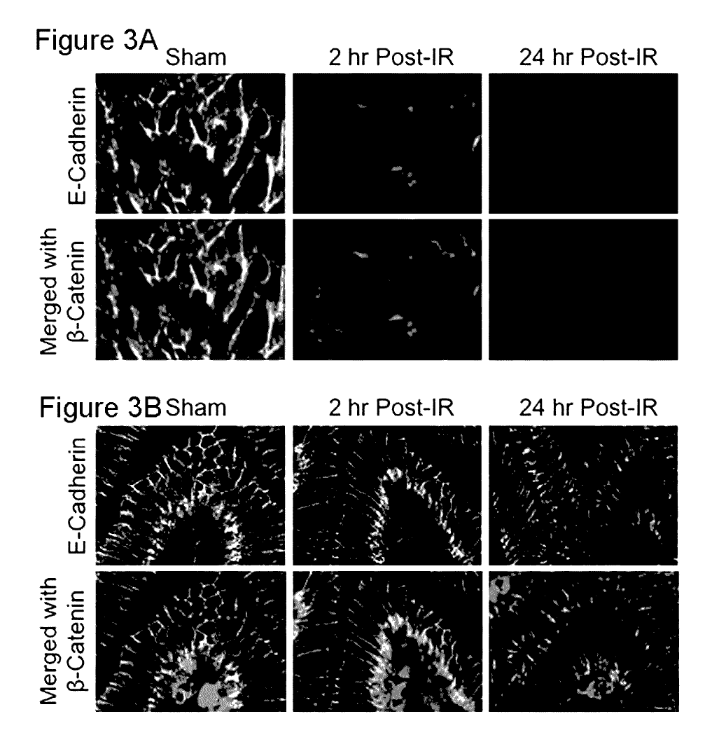

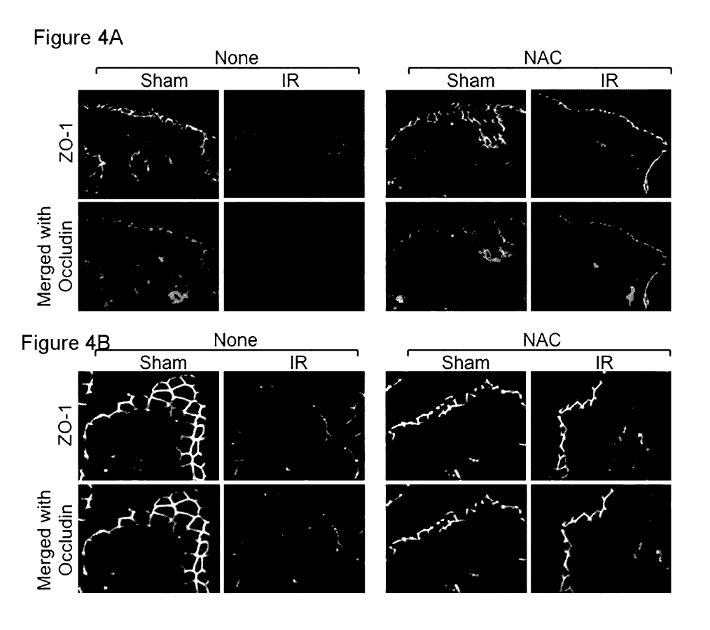

E-cadherin and β-catenin are the principal components of the epithelial AJs. Interaction between E-cadherin and β-catenin plays a role for the assembly and maintenance of AJs. Confocal microscopy showed that these two proteins are co-localized at the intercellular junctions of colonic (FIG. 3A) and ileal (FIG. 3B) epithelium. Radiation induced a redistribution of E-cadherin and β-catenin from the colonic epithelial junctions as early as 2 hours post-irradiation, and the damage sustained at least for 24 hours post-irradiation (FIG. 3A). There was a slight loss of E-cadherin and β-catenin in the epithelial junctions of ileum (FIG. 3B), but the effect was much less severe compared to that in colonic epithelium.

1.2.3 NAC Feeding Attenuates Radiation-Induced Disruption of TJs, Actin Cytoskeleton and AJs

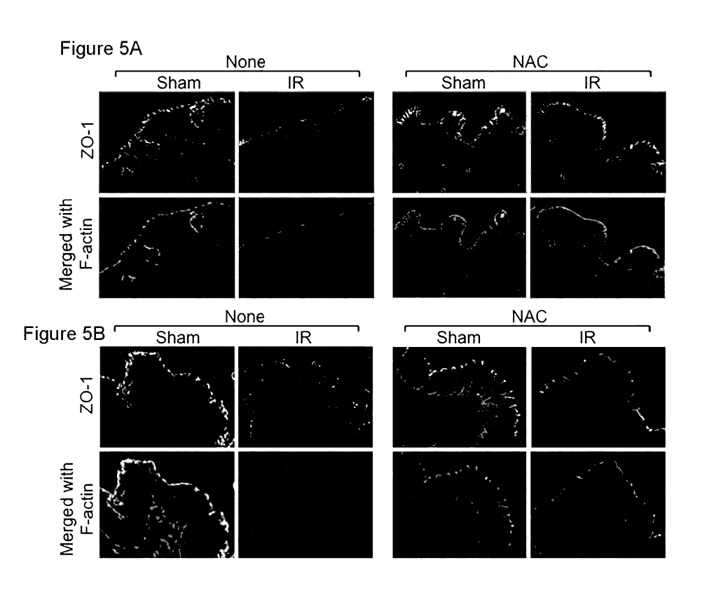

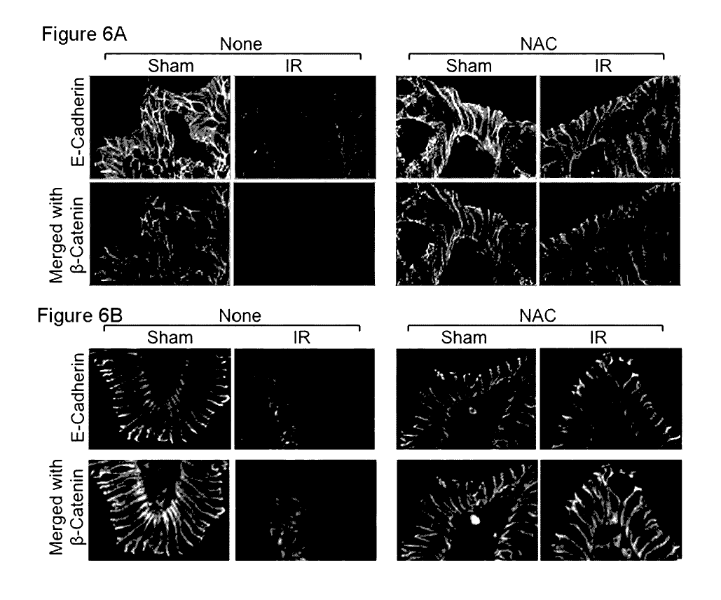

NAC is an antioxidant that acts by restoring cellular protein thiols from oxidative depletion. Prophylactic treatment of mice with 20 mM NAC in a liquid diet for 5 days prior to irradiation resulted in almost a complete attenuation of radiation-induced redistribution of occludin and ZO-1 from the intercellular junctions in colon (FIG. 4A) and ileum (FIG. 4B). NAC treatment also blocked radiation-induced redistribution of Cldn-3 in colon (FIG. 5A) and ileum (FIG. 5B). Radiation-induced loss of F-actin in the colon (FIG. 5A) and ileum (FIG. 5B) was prevented by NAC treatment; the protection was almost complete in the colon, but the actin cytoskeleton in the ileum was only partially protected. Radiation-induced redistribution of E-cadherin and β-catenin from the intercellular junctions of epithelium in the colon (FIG. 6A) and ileum (FIG. 6B) was also absent in NAC-fed mice.

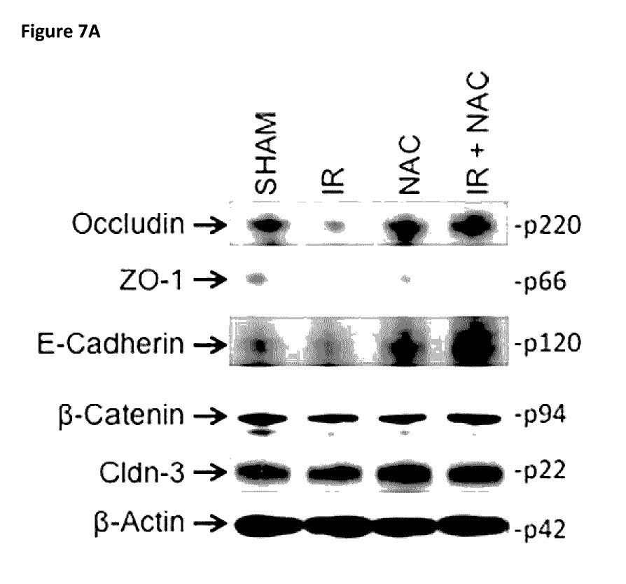

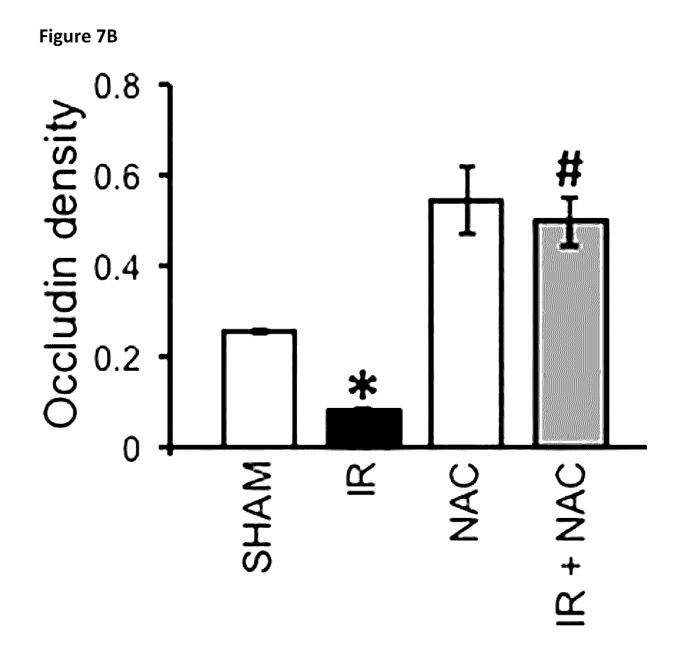

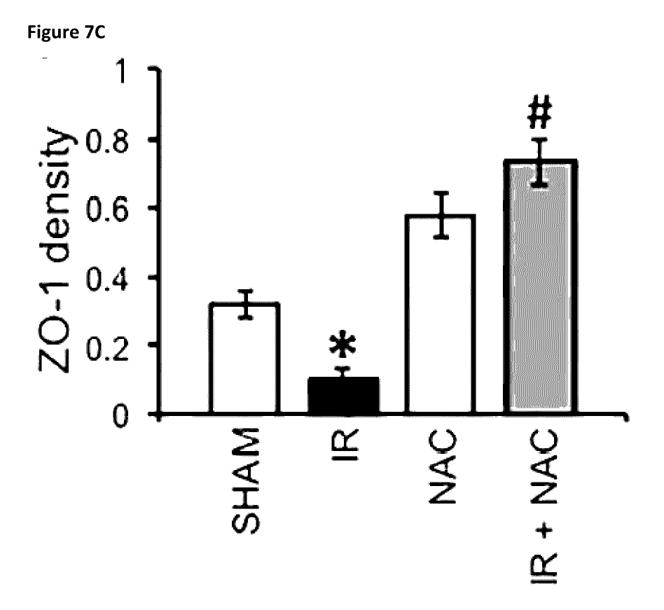

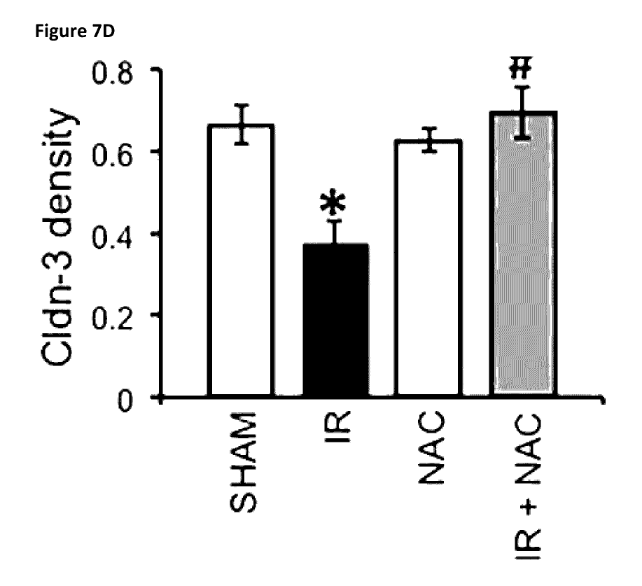

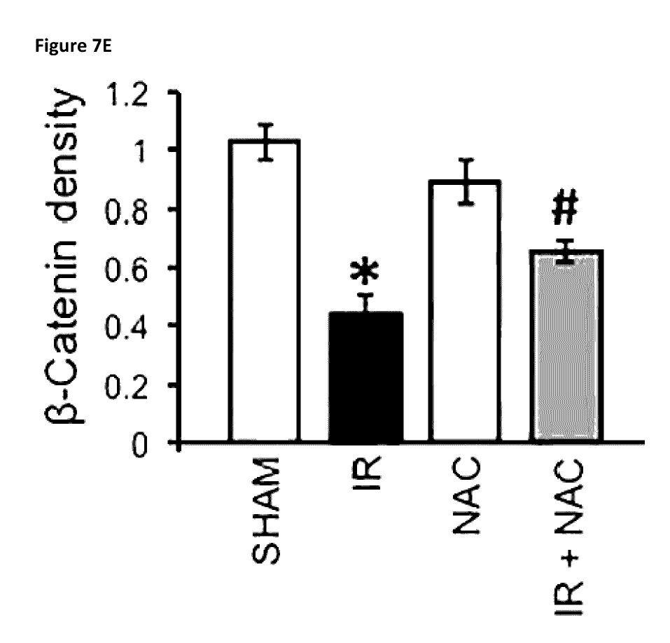

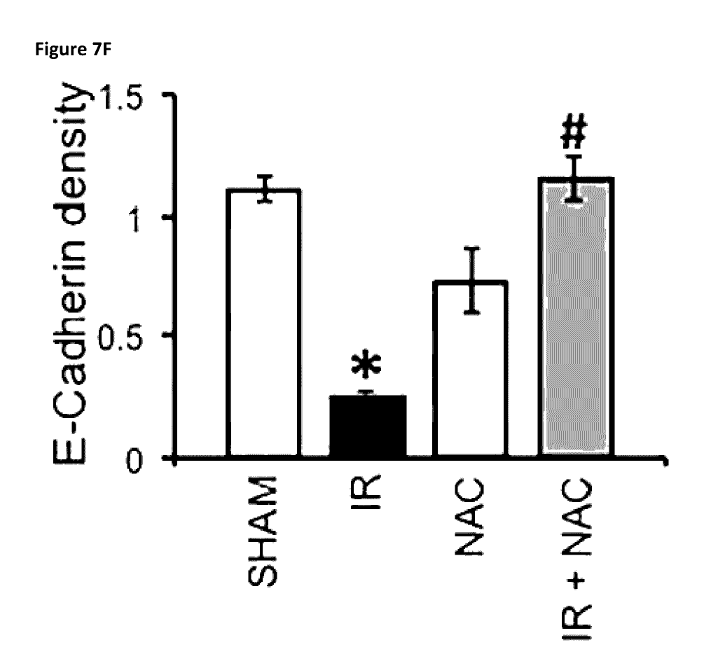

TJ and AJ protein complexes are attached to the actomyosin belt at the apical end of epithelial cells, and therefore, TJ and AJ proteins are pulled down along with the actin-rich detergent-insoluble fractions (Rao, R. K.; Basuroy, S.; Rao, V. U.; Karnaky Jr, K. J.; Gupta, A. Tyrosine phosphorylation and dissociation of occludin-ZO-1 and E-cadherin-beta-catenin complexes from the cytoskeleton by oxidative stress. Biochem J 368:471-481; 2002.). The level of TJ and AJ proteins in the detergent-insoluble fractions of epithelial cells is an excellent indicator of the integrity of TJ and AJ. Immunoblot analysis showed that radiation induced a loss of detergent-insoluble fractions of TJ and AJ proteins, and that NAC treatment blocked this effect of radiation (FIG. 7A). Densitometric analysis of specific bands confirmed a significant reduction of the levels of detergent-insoluble fractions of occludin (FIG. 7B), ZO-1 (FIG. 7C), Cldn-3 (FIG. 7D), E-cadherin (FIG. 7E) and β-catenin (FIG. 7F). These results are complementary to the confocal microscopic data described above.

1.2.4 Radiation Increases Intestinal Mucosal Permeability, and NAC Feeding Prevents this Effect of Radiation

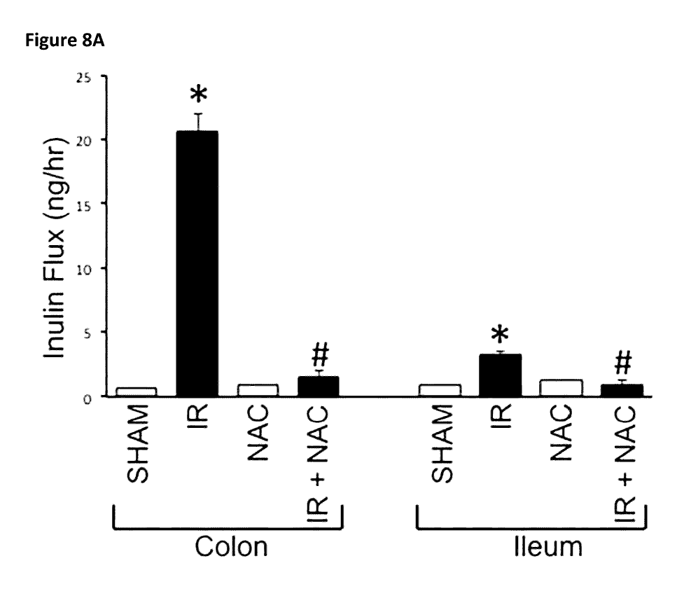

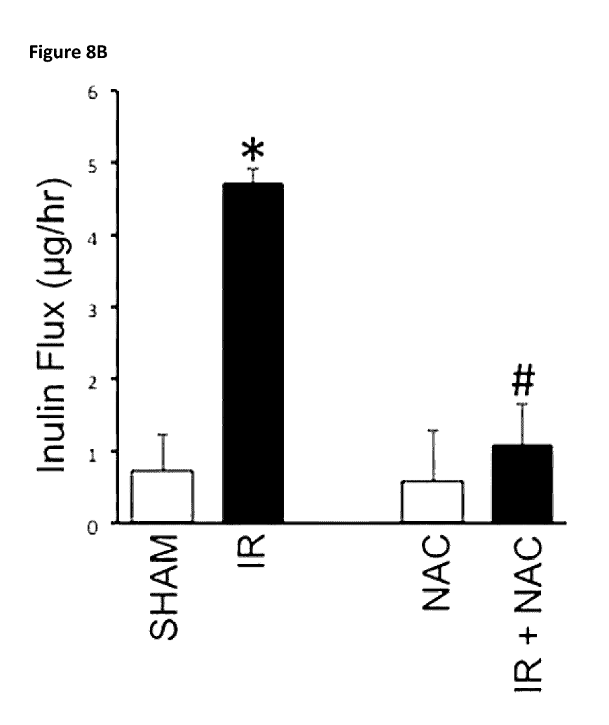

Measurement of vascular-to-luminal flux of FITC-inulin in vivo showed that irradiation significantly increased mucosal permeability to FITC-inulin, nearly 30 folds in colon. NAC treatment effectively attenuated radiation-induced increase in colonic mucosal permeability (FIG. 8A). Radiation increased inulin permeability also in the ileum, although the effect was much lower compared to that in colon (FIG. 8A). Radiation-induced increase in inulin permeability in the ileum was also blocked by NAC supplementation in diet. NAC, by itself, showed no significant influence on the mucosal permeability in colon or ileum.

Analysis of luminal-to-mucosal permeability to FITC-inulin in vitro in isolated loops of colon showed similar effects of radiation and NAC. The amount of inulin absorbed from the lumen was several folds high in the colonic loop prepared from irradiated mice compared to that in loops prepared from Sham-treated mice (FIG. 8B). This effect of radiation was absent in colonic loops prepared from NAC-fed, irradiated mice.

1.2.5 Ionizing Radiation Depletes Protein Thiols in Mouse Intestine, and NAC Feeding Prevents this Effect of Radiation.

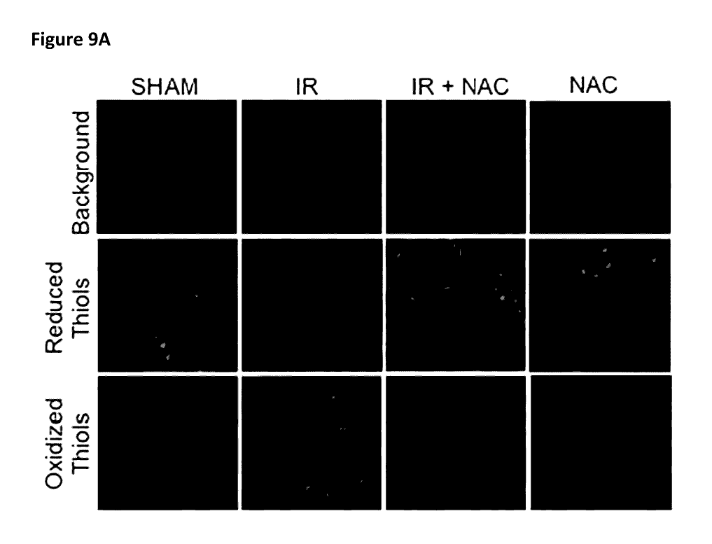

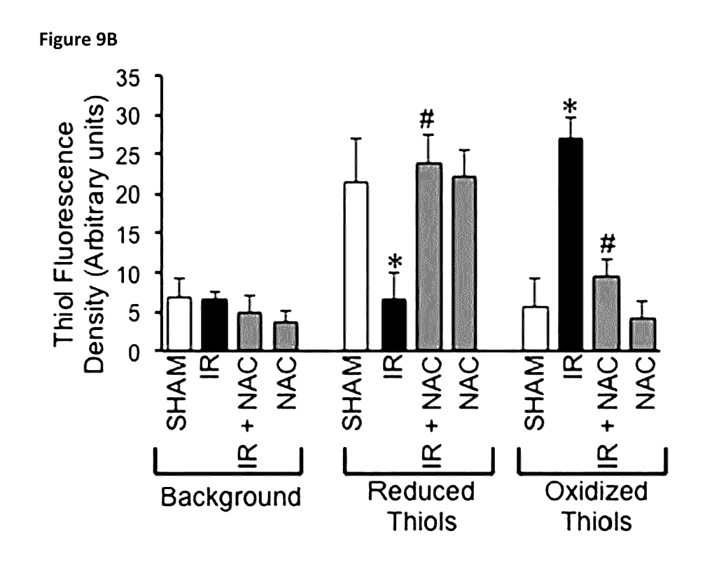

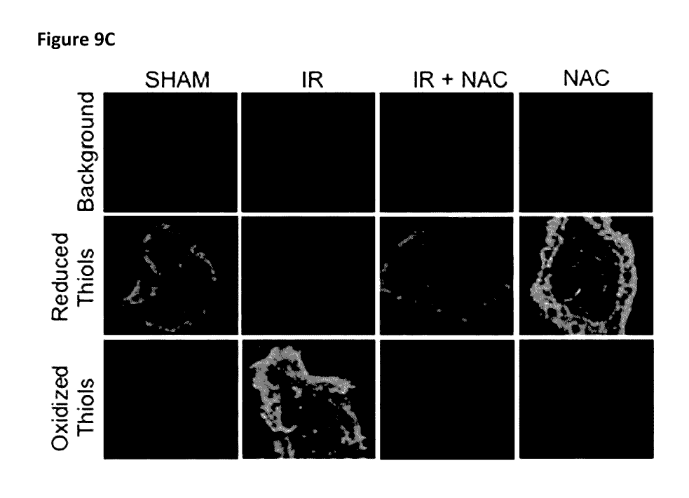

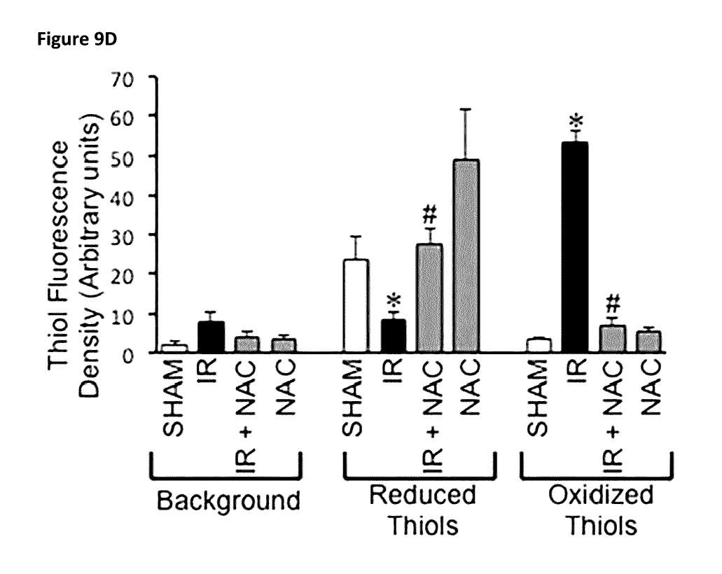

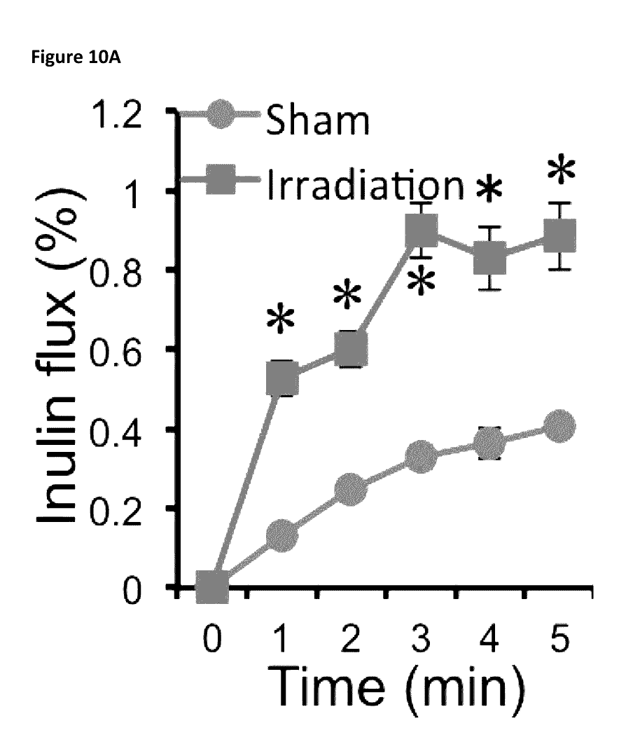

Here we show that radiation dramatically reduced the stain for reduced protein thiols with a concomitant increase in the stain for oxidized protein thiols in both colon (FIG. 9A) and ileum (FIG. 9C). These effects of radiation were absent in colon and ileum of NAC fed mice. Densitometric analysis of fluorescence for reduced and oxidized protein thiols in samples from different mice confirmed that radiation significantly converts reduced protein thiols into oxidized protein thiols in colon (FIG. 9B) and ileum (FIG. 9D), and that NAC feeding attenuates this effect of radiation.

### EXAMPLE 2

### Protection of Colonic Barrier Function by LPA2 Receptor Agonists RP-1 and LPA from Irradiation

2.1.1 Radiation Induces Redistribution of TJ Proteins and Causes Barrier Dysfunction in m-ICC12 and Caco-2 Cell Monolayers

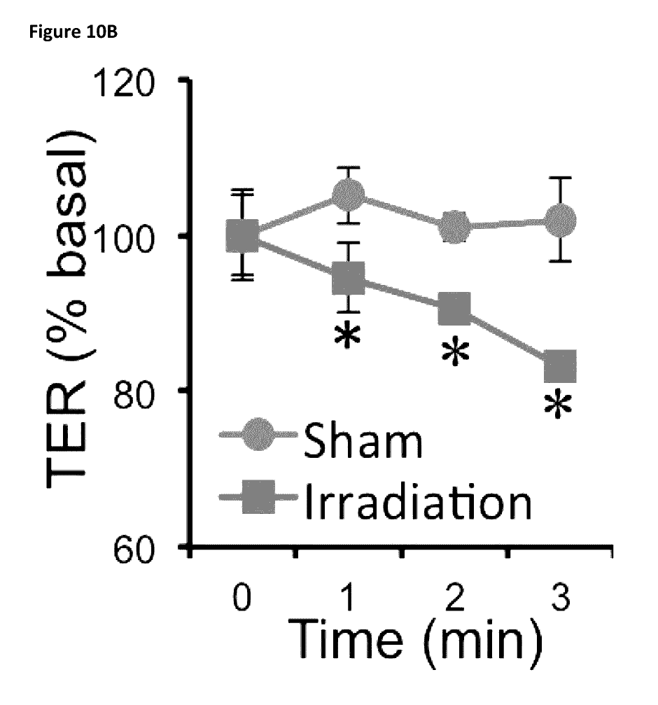

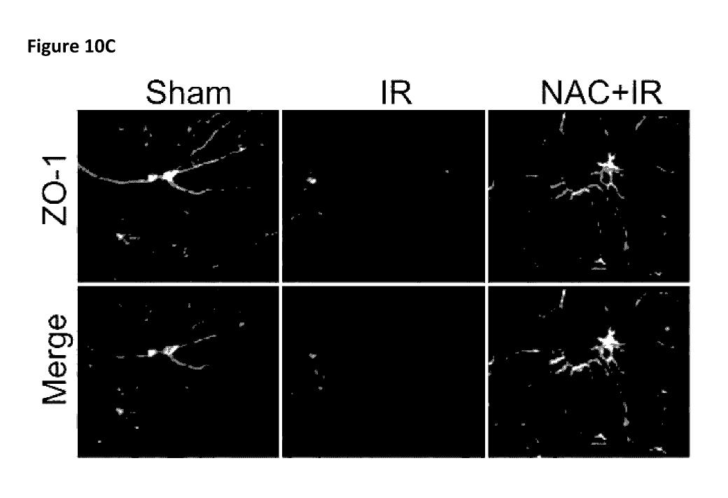

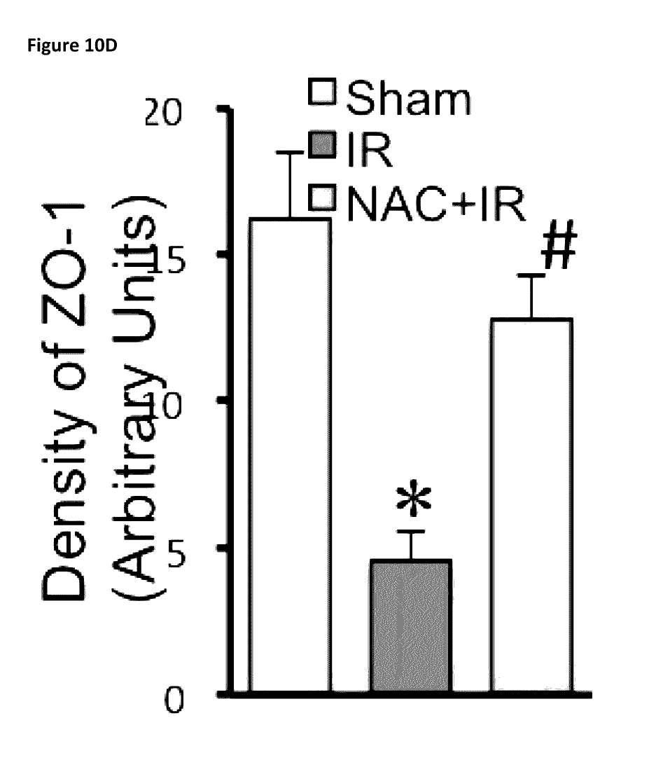

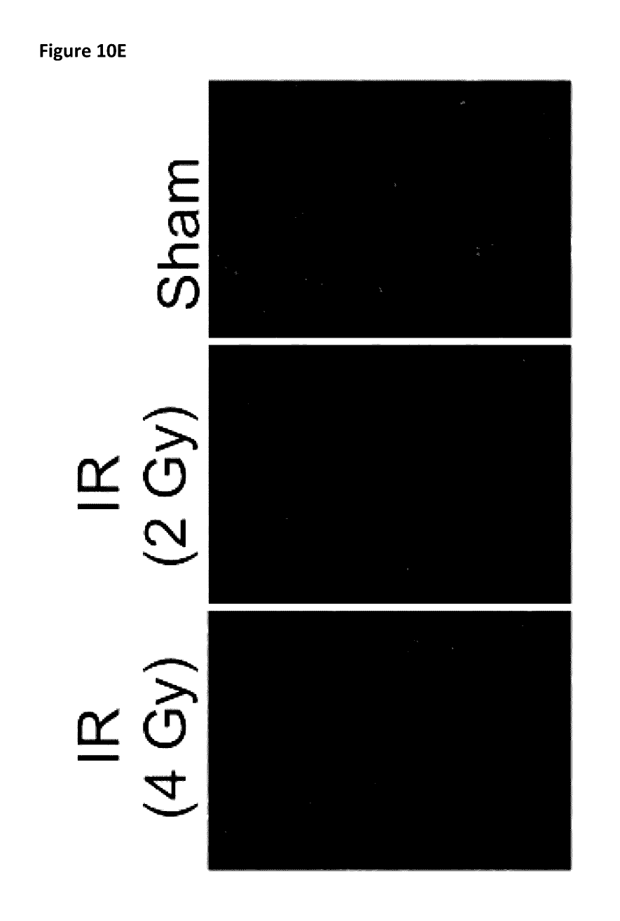

The effects of IRR on TJ and AJ integrity in vivo in the above-described study do not answer whether the radiation effect was due to a direct effect on the epithelial cells. Therefore, we evaluated the effect of radiation on barrier function and TJ proteins in Caco-2 and m-ICC12 cell monolayers in vitro. Barrier function was evaluated by measuring transepithelial electrical resistance (TER) and transepithelial flux of FITC-inulin in cell monolayers grown on transwell inserts. Cell monolayers were also fixed and stained for occludin and ZO-1 for confocal microscopy. Results presented in FIGS. 10A, 10B, 10C, 10D, and 10E show that radiation (4 Gy) increases inulin permeability (FIG. 10A) and reduces TER (FIG. 10B) in Caco-2 cell monolayers; this loss of barrier function was associated with a loss of junctional distribution of occludin and ZO-1, indicating a loss of TJ integrity (FIG. 10C). Radiation-induced loss of junctional occludin and ZO-1 was significantly blocked by pretreatment of cells with NAC (10 mM) for one hour, suggesting that oxidative stress mediates radiation-induced loss TJ integrity in Caco-2 cell monolayers as well.

Caco-2 cells have been widely used to study barrier function. However, this is a transformed cell line. So, we tested the radiation effect in m-ICC12 cell monolayers, a non-transformed mouse intestinal epithelial cell line. Results show that radiation (2-4 Gy) induced rapid redistribution of ZO-1 from the intercellular junctions into the intracellular compartment (FIG. 10E). These data further support a direct effect of radiation on the intestinal epithelium. These in vitro models can be used to understand the cellular and molecular mechanisms involved in radiation injury and in the action of radiomitigators.

2.1.2 Radiation Activates Cofilin and Reorganizes Actin Cytoskeleton in Mouse Colon and Caco-2 Cell Monolayers

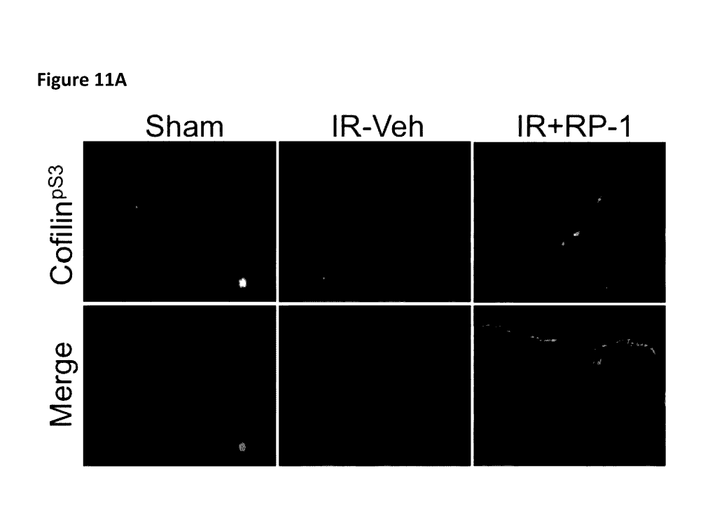

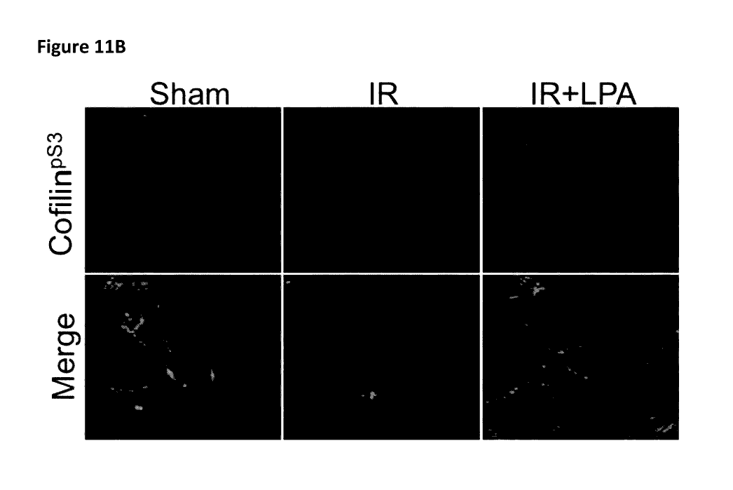

Here we examined the organization of actin cytoskeleton in control and irradiated cell monolayers. Cofilin is an actin severing protein involved in destabilization of actin cytoskeleton16-19. Cofilin activity is regulated by phosphorylation on Ser-3. Phospho-cofilin is inactive and its activity is controlled by Ser-kinases, such as LIM kinase. Therefore, we examined the level of cofilinpS3 in irradiated mouse colon and Caco-2 cells. Results presented in FIGS. 11A and 11B show that the actin organization is visibly altered in the irradiated mouse colon (2 hour post-IRR) (FIG. 11A), which was associated with a reduction in the levels of cofilinpS3 (FIG. 11B). Similarly, IRR-induced reduction of cofilinpS3 was observed in Caco-2 cell monolayers (FIGS. 11C & 11D). These results indicate that radiation can activate cofilin and induce actin remodeling. Taken together, these processes provide a plausible mechanism of radiation-induced TJ and AJ disruption, barrier dysfunction, and endotoxemia.

2.1.3 RP-1 Blocks TBI-Induced Redistribution of TJ and AJ Proteins in Mouse Colon.

Studies were performed to evaluate the effect of RP-1 on radiation-induced redistribution of TJ and AJ proteins in mouse colon in vivo. Mice were subjected to TBI (4 Gy); in some groups, animals received a one-time injection of RP-1 (0.1 mg/kg) 30 min prior to IRR. At 2 hours post-IRR, cryosections of colon were stained for TJ and AJ proteins, images collected by confocal microscopy and fluorescence measured by densitometric analysis.

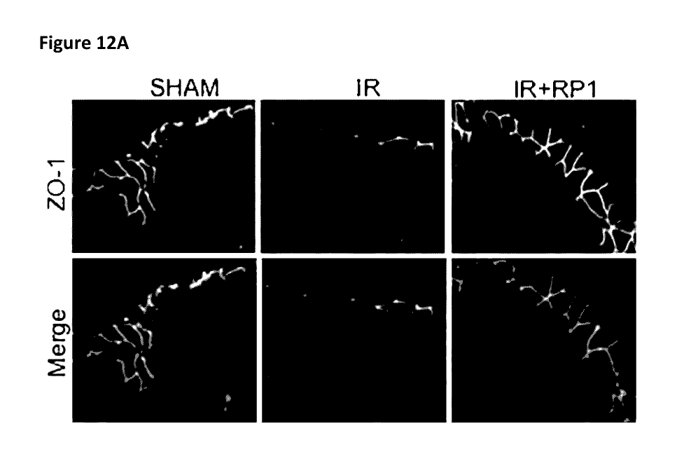

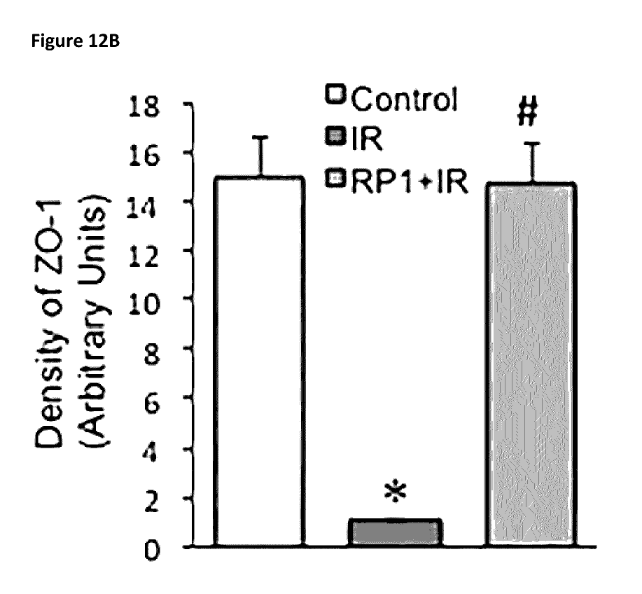

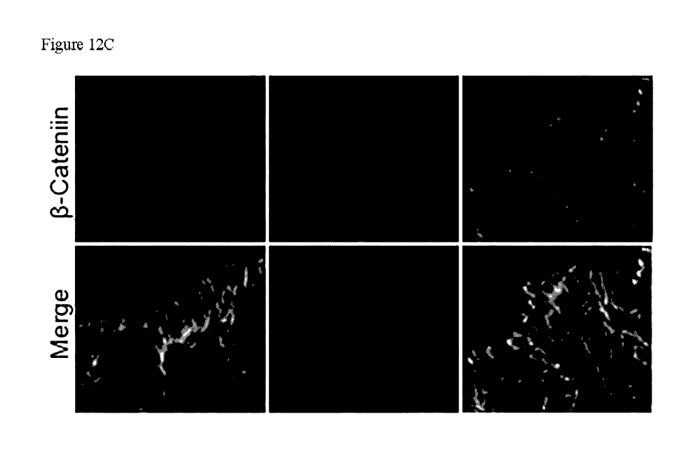

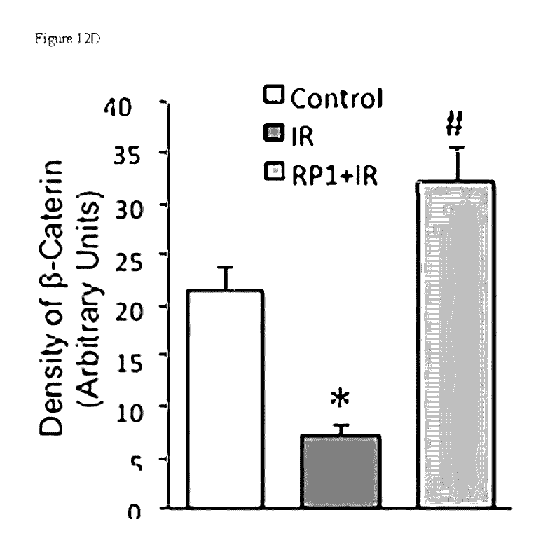

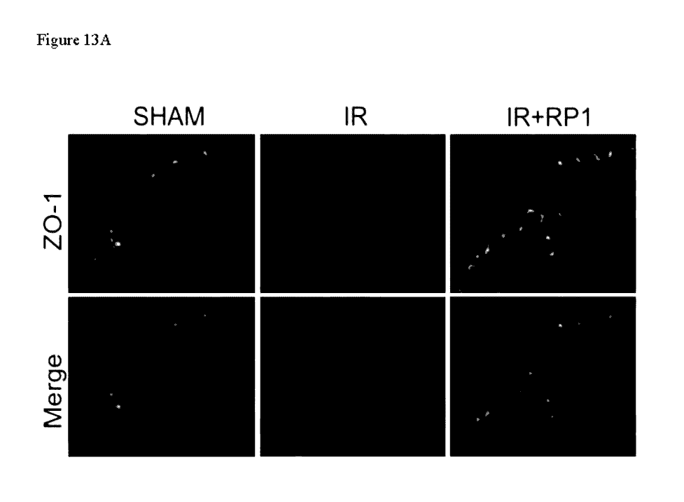

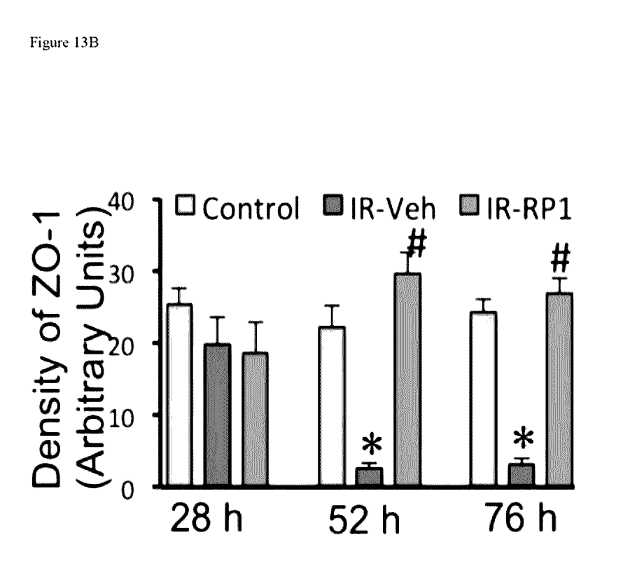

Results presented in FIGS. 12A and 12B show that RP1 treatment almost completely blocked TBI-induced redistribution of occludin and ZO-1 from the junctions of colonic epithelium (FIGS. 12A & 12B). Similarly, RP-1 blocked radiation effect on AJ proteins (FIGS. 12C & 12D). These results indicate that RP-1 protects colonic epithelial TJ and AJ from radiation.

2.1.4 RP-1 Mitigates PBI-BM5-Mediated Redistribution of TJ and AJ Proteins in Mouse Colon.

To determine the potential mitigating effect of RP-1 we administered RP-1 at a dose of 0.1 mg/kg BW, i.p., every 12 hours starting 24 hours post-IRR in PBI-BM5 model. At 28-100 hours post-IRR the cryosections of colon were stained for TJ and AJ proteins, and the fluorescence was measured by densitometric analysis.

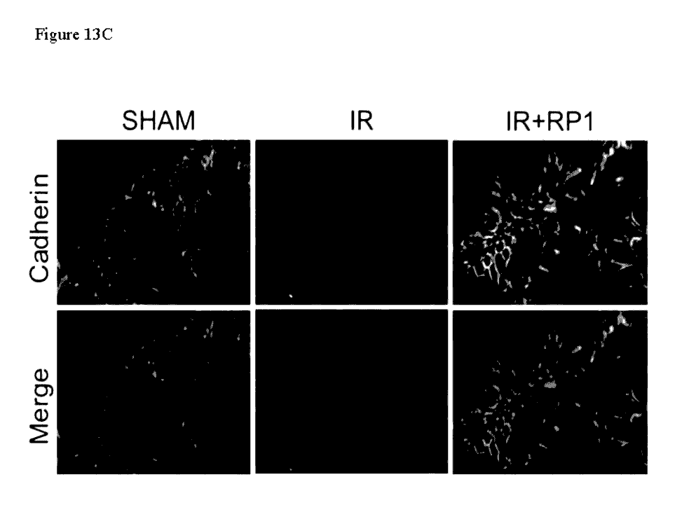

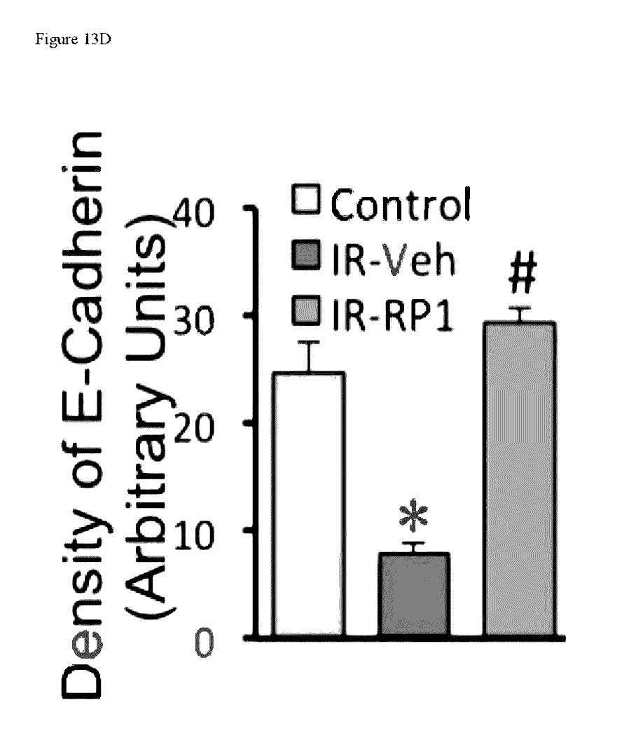

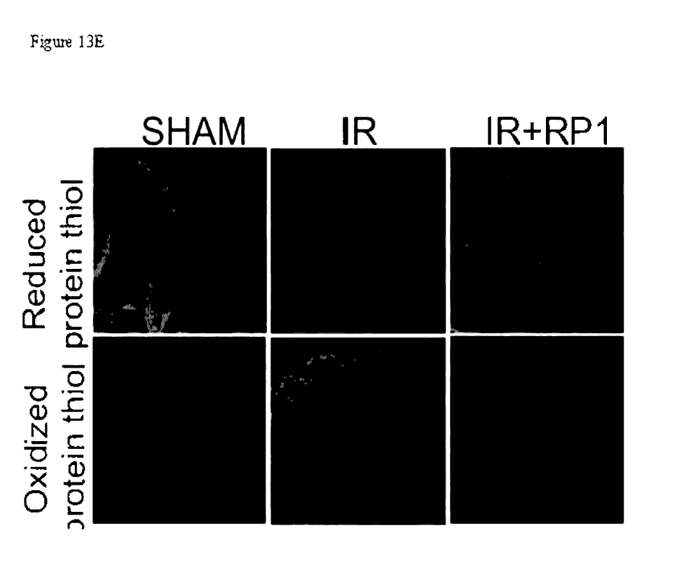

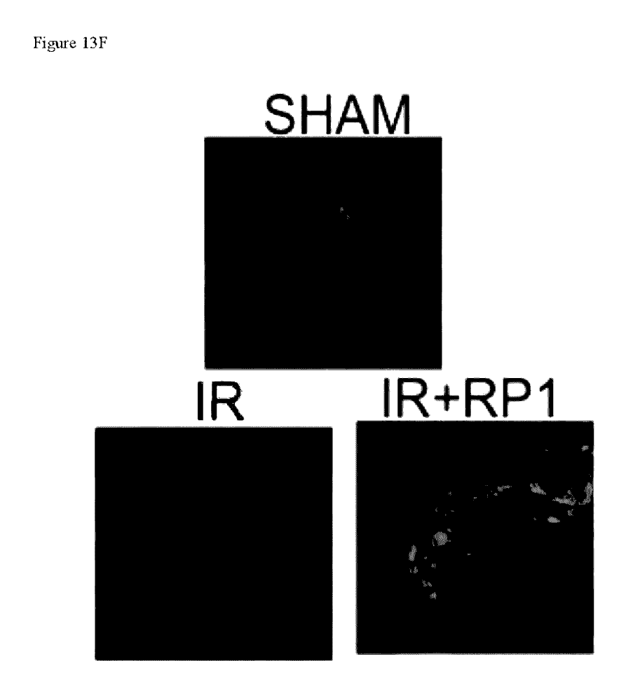

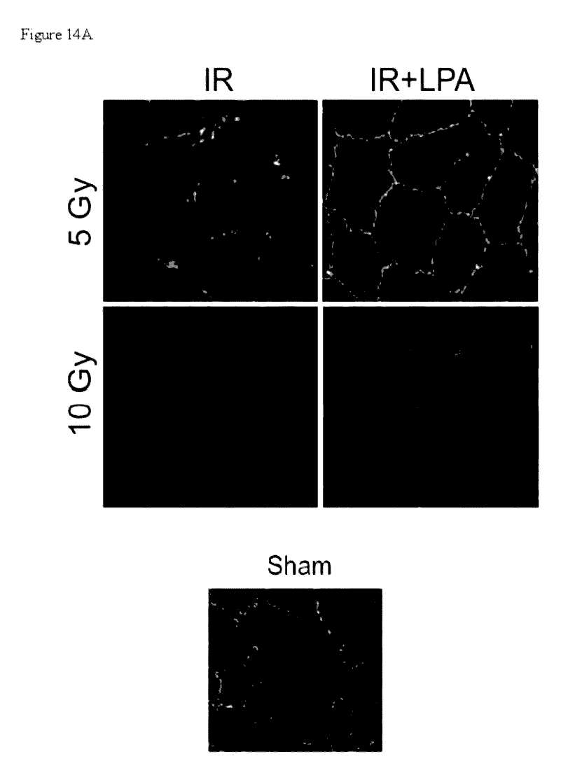

The results presented in FIG. 13A-13F show that a redistribution of occludin and ZO-1 occurred at 52 hours post-IRR, but not at 28 hours (FIGS. 13A & 13B), and sustained at least until 100 hours (100 hr data not shown). This effect of PBI on colonic epithelial TJ is slower than that of TBI, in which TJ protein redistribution occurred at 2 hours post-IRR. Data suggest that a different mechanism in addition to epithelial oxidative stress may be involved in TBI-induced TJ and AJ reorganization. RP-1 treatment restored junctional distribution of occludin and ZO1 in colon and this effect continued for at least 100 hours (FIGS. 13A & 13B). Data also show that RP-1 restored GI-ARS-induced redistribution of E-cadherin and β-catenin from the colonic epithelial junctions (FIGS. 13C & 13D). FIG. 13E shows that RP-1 mitigates PBI-BM5-induced oxidation of protein thiols, and FIG. 10F indicates that IRR reduces the levels of Nrf2, which was mitigated by RP-1 treatment.

2.1.5 LPA Attenuates Radiation-Induced Redistribution of TJ Proteins in m-ICC12 and Caco-2 Cells.

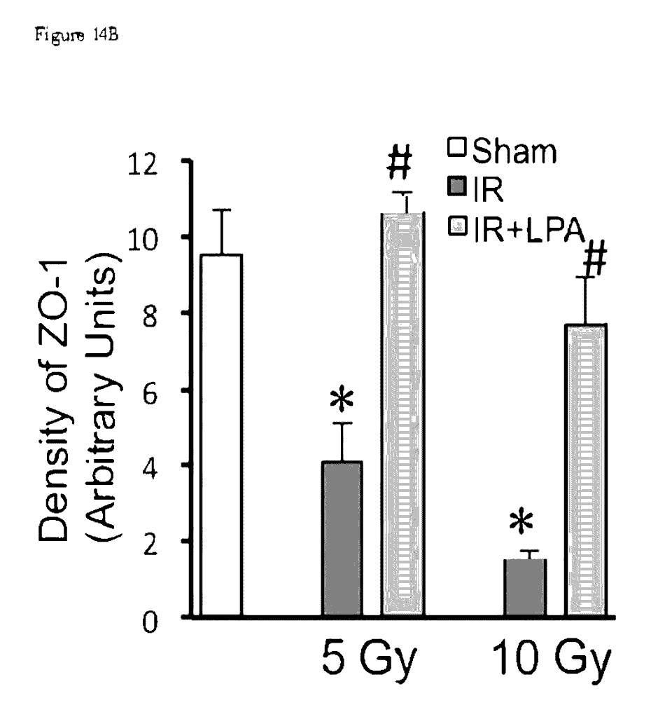

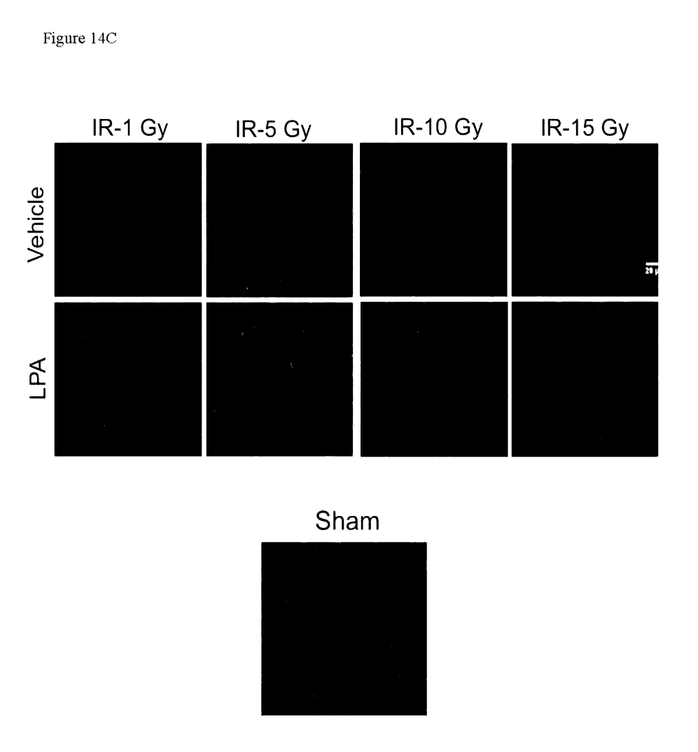

To determine whether the RP-1 effect on mouse colonic TJ and AJ is a direct effect on the epithelial cells we tested the effect of LPA treatment (10 μM) administered 10 min post-IRR on radiation-induced redistribution of TJ proteins. Results presented in FIG. 14A-14C show that LPA attenuates IRR-induced redistribution of occludin and ZO-1 (FIGS. 14A & 14B). The effect of LPA was also tested in m-ICC12 cells at varying doses of radiation. LPA blocked IRR-induced redistribution of ZO-1 (FIG. 14C). These data indicate that LPA effect involves direct interaction with epithelial cells. These models can be used for further mechanistic studies.

### EXAMPLE 3

### Protection of Alcohol-Induced Colonic Barrier Function by LPA2 Receptor Agonists RP-1 and LPA

3.1.1 RP-1 Administration Blocks Ethanol-Induced Increase in Inulin Permeability in Mouse Intestine.

Gut barrier dysfunction and increased endotoxin flux from the colonic lumen into mesenteric circulation are associated with alcoholic liver disease ALD. A significant body of evidence indicates that endotoxins play a crucial role in the pathogenesis of ALD. Therefore, factors that prevent alcohol-induced gut barrier dysfunction and endotoxemia have potential therapeutic benefit in the prevention/mitigation of ALD. In this study we evaluated the effect of RP1 on alcohol-induced gut barrier dysfunction and fatty liver in mouse model of chronic alcohol consumption. Two different models of alcoholic administration were used. In the first model (Chronic+Binge) mice were fed 5% ethanol in Lieber-DiCarli liquid diet for 10 days followed by one time gavage of ethanol (5 g/kg BW). In the second model (Chronic) mice were fed ethanol (1-6%) in Lieber-DiCarli liquid diet for 4 weeks. RP1 was injected subcutaneously (0.2 mg/kg BW). The control animals were pair fed with isocaloric diet without ethanol. Intestinal mucosal permeability in vivo was evaluated by measuring vascular-to-luminal flux of FITC-inulin. FITC-inulin was administered by tail vein injection. One hour after injection, fluorescence was measured in the luminal flushings of different segments of intestine and plasma. Triglyceride content was measured in liver extracts.

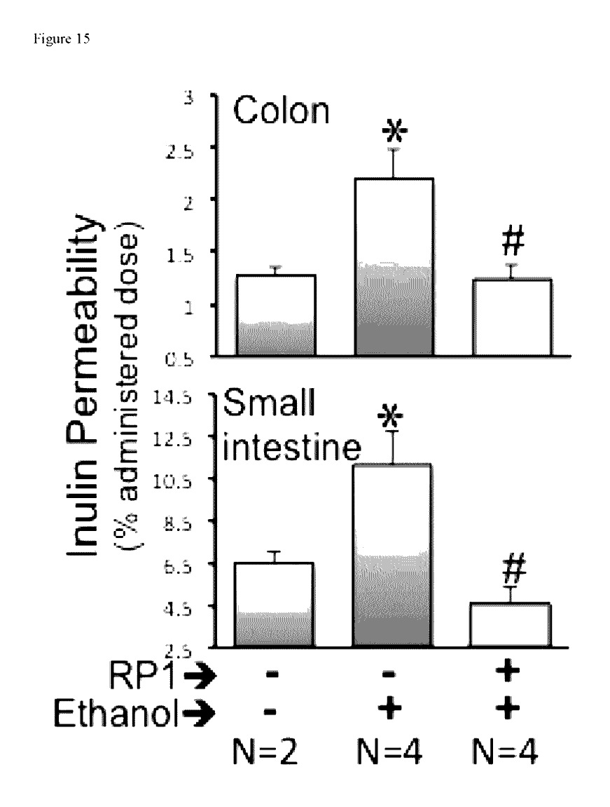

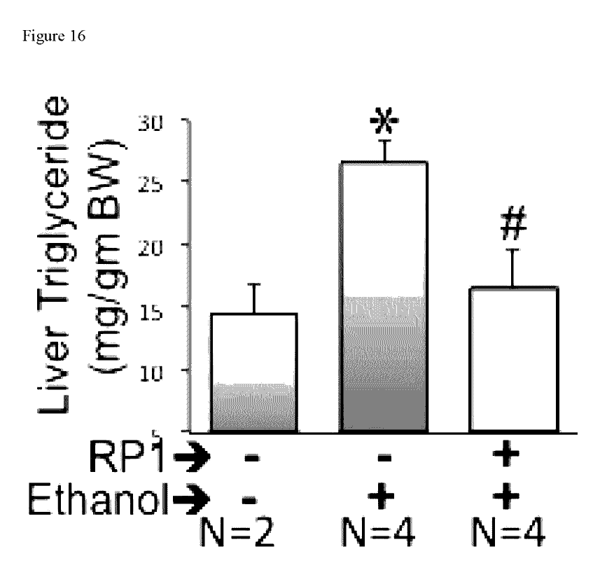

As shown in FIG. 15, Chronic+Binge ethanol feeding significantly increased inulin permeability in colon and small intestine (FIG. 15), and RP1 administration significantly attenuated this alcohol-induced increase in inulin permeability, suggesting that RP1 prevents alcohol-induced disruption of epithelial barrier function. This study also showed that RP1 administration significantly reduced Chronic+Binge alcohol-induced accumulation of triglyceride deposition in the liver (FIG. 16). These results indicate that RP1 may have potential therapeutic benefit in prevention/treatment of ALD.

3.1.2 LPA2 Deficiency Promotes Ethanol-Induced Colonic Inulin Permeability and Fatty Liver.

RP1 is a highly selective agonist of LPA2 receptor and its role in prevention of alcohol-induced gut barrier dysfunction is likely involves LPA2 receptor activation. To determine the role of LPA2 receptor we evaluated gut barrier function and fatty liver in wild type and LPA2 deficient mice. Wild type and LPA2 knock mice were fed ethanol in Lieber DiCarli liquid diet as described above. Inulin permeability in vivo and liver triglyceride levels were measured.

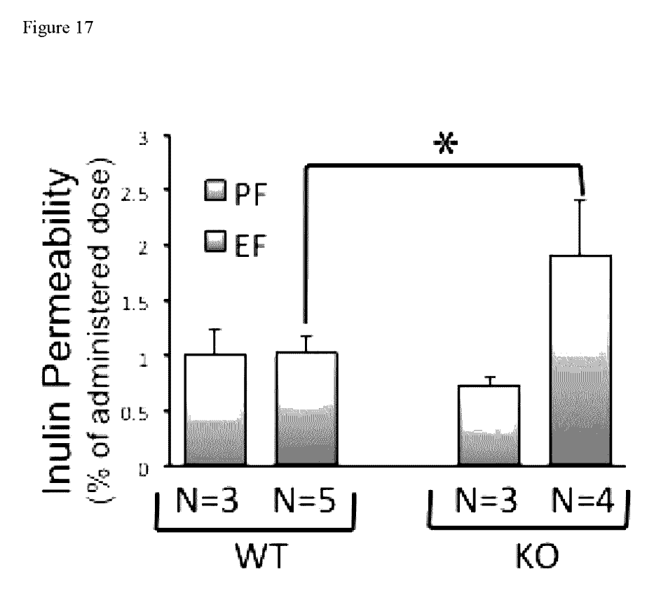

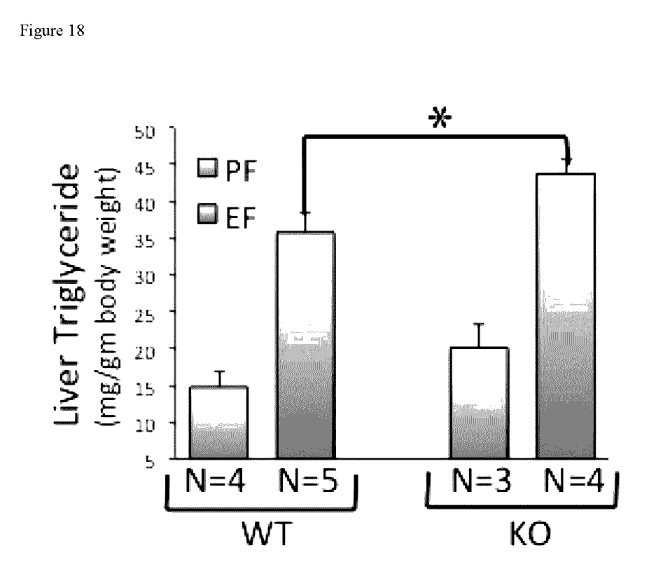

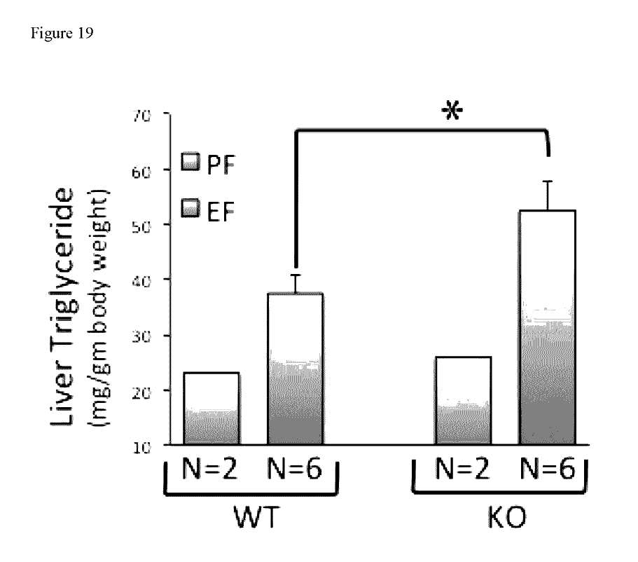

Results show that, in the Chronic+Binge model, the wild type mouse strains were quite resistant to alcohol-induced gut barrier dysfunction. But, alcohol significantly increased inulin permeability in the colon of LPA2-KO mice (FIG. 17). Alcohol-induced triglyceride deposition in the liver was significantly greater in LPA2-KO mice (FIG. 18). Alcohol-induced liver triglyceride deposition was greater in LPA2-KO mice also in chronic ethanol feeding model (FIG. 19). These results show that endogenous LPA and LPA2 receptor activation may have a protective effect in alcoholic tissue injury.

3.1.3 LPA Attenuates Radiation-Induced Barrier Dysfunction in Caco-2 Cell Monolayers

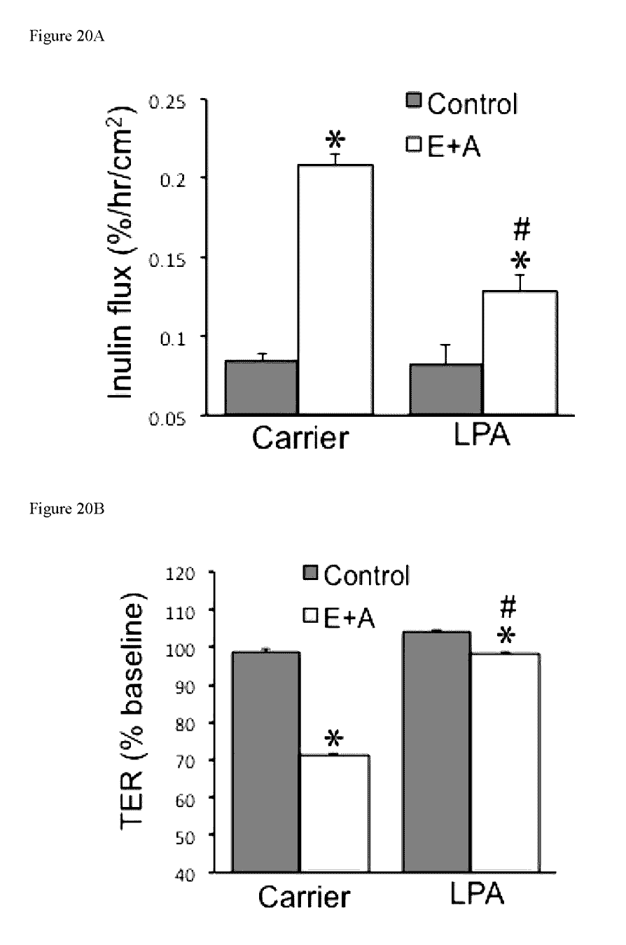

To determine the direct of LPA on the intestinal epithelium and protection of barrier function, we evaluated the effect of LPA treatment on barrier function in Caco-2 cell monolayers in vitro. Caco-2 cell monolayers grown on transwell inserts were preincubated with LPA 30 min prior to exposure to 0.5% ethanol and 400 μM acetaldehyde (the toxic metabolic product of ethanol). At 4 hours after ethanol+acetaldehyde treatment, barrier function was evaluated by measuring transepithelial electrical resistance (TER) and transepithelial flux of FITC-inulin. Results show that LPA treatment significantly reduced ethanol+acetaldehyde-induced increase in inulin permeability (FIG. 20A) and decrease in TER (FIG. 20B). This study demonstrates that LPA has a direct impact on the intestinal epithelium and preserves the barrier function in the presence of ethanol and acetaldehyde.

What is claimed is:

1. A method of treating or preventing colonic barrier dysfunction in a human, comprising administering to the human in need thereof a pharmaceutically effective amount of an LPA2 receptor agonist.

2. The method of claim 1, wherein the LPA2 receptor agonist is RP-1.

3. The method of claim 1, wherein the LPA2 receptor agonist is LPA.

4. The method of claim 1, wherein the colonic barrier dysfunction is caused by irradiation.

5. The method of claim 1, wherein colonic barrier dysfunction is caused by alcohol consumption.

6. The method of claim 1, wherein the administration of the compound is prior to the occurrence of the colonic barrier dysfunction.

7. The method of claim 1, wherein the administration of the compound is after the occurrence of the colonic barrier dysfunction.

8. A medicament for treating or preventing colonic barrier dysfunction in a human, comprising an LPA2 receptor agonist.

9. The medicament of claim 8, wherein the colonic barrier dysfunction is caused by irradiation.

10. The medicament of claim 8, wherein the colonic barrier dysfunction is caused by alcohol consumption.

11. A method of treating or preventing colonic barrier dysfunction in a human, comprising administering to the human in need thereof a pharmaceutically effective amount of N-Acetyl L-Cysteine.

12. The method of claim 11, wherein the colonic barrier dysfunction is caused by irradiation.

13. The method of claim 11, wherein colonic barrier dysfunction is caused by alcohol consumption.

14. The method of claim 11, wherein the administration of the compound is prior to the occurrence of the colonic barrier dysfunction.

15. The method of claim 11, wherein the administration of the compound is after the occurrence of the colonic barrier dysfunction.

16. A medicament for treating or preventing colonic barrier dysfunction in a human, comprising N-Acetyl L-Cysteine.

17. The medicament of claim 16, wherein the colonic barrier dysfunction is caused by irradiation.

18. The medicament of claim 16, wherein the colonic barrier dysfunction is caused by alcohol consumption.
